(19) United States
(12) Patent Application Publication
(10) Pub. No.: US 2009/0253693 A1
(43) Pub. Date: Oct. 8, 2009

(54) 2H-BENZO[b][1,4]OXAZIN-3(4H)-ONE DERIVATIVES FOR USE AS STEAROYL CoA DESATURASE INHIBITORS

(76) Inventors: Dmitry Koltun, Foster City, CA (US); Jeff Zablocki, Los Altos, CA (US); Natalya Vasilevich, Moscow (RU); Vasily Migulin, Moscow (RU)

Correspondence Address: CV THERAPEUTICS, INC., 3172 PORTER DRIVE, PALO ALTO, CA, 94304, US

(21) Appl. No.: 12/417,854

(22) Filed: Apr. 3, 2009

Related U.S. Application Data
(60) Provisional application No. 61/042,983, filed on Apr. 7, 2008.

(51) Int. Cl. A61K 31/538; C07D 265/36; A61P 9/00

(52) U.S. Cl. 514/230.5; 544/105

(57) ABSTRACT

The present invention discloses 2H-benzo[b][1,4]oxazin-3(4H)-one derivatives for use as inhibitors of stearoyl-CoA desaturase having the structure of Formula I:

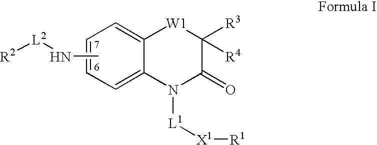

The compounds are useful in treating and/or preventing various human diseases, mediated by stearoyl-CoA desaturase (SCD) enzymes, especially diseases related to abnormal lipid levels, cardiovascular disease, diabetes, obesity, metabolic syndrome and the like.

This application claims priority to U.S. Provisional Patent Application Ser. No. 61/042,983, filed Apr. 7, 2008, the entirety of which is incorporated herein by reference.

### FIELD OF THE INVENTION

The present invention relates generally to the field of inhibitors of stearoyl-CoA desaturase, such as 2H-benzo[b][1,4]oxazin-3(4H)-one derivatives, and uses for such compounds in treating and/or preventing various human diseases, mediated by stearoyl-CoA desaturase (SCD) enzymes, especially diseases related to elevated lipid levels, cardiovascular disease, cancer, diabetes, obesity, metabolic syndrome and the like.

### BACKGROUND

Stearoyl CoA desaturases (SCD's) are Δ9 fatty acid desaturases. The mammalian enzymes are localized to the endoplasmic reticulum and require molecular O₂ and NADH to desaturate saturated fatty acids at the Δ9 position and generate monounsaturated fatty acids and water in the process. The primary substrates for these enzymes are the acyl-CoA derivatives of stearic (C18) and palmitic acids (C16) with the major reaction being the conversion of stearic acid to oleic acid (C18:1). Depending on the species, 2-4 highly homologous isoforms of SCD exist differing primarily in tissue distribution.

The best characterized SCD isozyme is SCD1 which is primarily found in liver, adipose and skeletal muscle. Deletion, mutation or inhibition of SCD1 in mice and rats results in decreased hepatic triglyceride secretion, decreased hepatic steatosis, resistance to weight gain and improvements in insulin sensitivity and glucose uptake (reviewed in Ntambi et al. (2004) Prog Lipid Res 43, 91-104; (2005), Prostaglandins Leukot. Essent. Fatty Acids 73, 35-41; and (2005) Obes. Rev. 6, 169-174). These studies combined with studies in humans showing correlations between surrogates for SCD activity and metabolic syndrome, diabetes and obesity strongly implicate SCD inhibition as a means to treat obesity, diabetes, hypertryglyceridemia and associated diseases and co-morbidities. Studies done using antisense oligonucleotide inhibitors have also demonstrated a correlation between SCD activity and obesity and the onset of diet-induced hepatic insulin resistance; see Jiang et al. (2005) J. Clin. Invest. 115:1030-1038G. and Gutiérrez-Juárez et al. (2006) J. Clin. Invest. 116:1686-1695.

The present invention presents compounds that are useful in inhibiting SCD activity and thus regulating lipid levels and lipid fatty acid composition. These compounds are useful in the treatment of SCD-mediated diseases such as diseases related to dyslipidemia and disorders of lipid metabolism, including, but not limited to diseases related to elevated lipid levels, cardiovascular disease, cancer, diabetes, obesity, metabolic syndrome and the like.

### SUMMARY OF THE INVENTION

It is an object of this invention to provide compounds that act as stearoyl-CoA desaturase inhibitors. Accordingly, in a first aspect, the invention relates to stearoyl-CoA desaturase inhibitor compounds having the structure of Formula I:

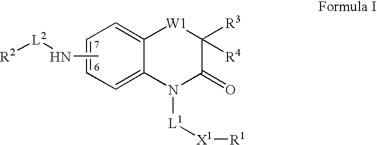

wherein
R¹ is hydrogen, optionally substituted C₁₋₂₀ alkyl, optionally substituted C₁₋₆ lower alkyl, optionally substituted C₃₋₂₀ cycloalkyl, optionally substituted C₂₋₂₀ alkenyl, optionally substituted C₂₋₂₀ alkynyl, optionally substituted C₁₋₂₀ alkoxy, optionally substituted C₁₋₆ alkoxy, optionally substituted mono- or bicyclic heterocyclyl, optionally substituted mono- or bicyclic aryl, or optionally substituted mono- or bicyclic heteroaryl;
R² is C₁₋₂₀ alkyl, optionally substituted mono- or bicyclic heterocyclyl, optionally substituted mono- or bicyclic aryl, or optionally substituted mono- or bicyclic heteroaryl;
R³ and R⁴ arc independently hydrogen, optionally substituted C₁₋₆ alkyl, optionally substituted C₂₋₆ alkenyl, optionally substituted C₂₋₆ alkynyl, optionally substituted cycloalkyl, optionally substituted mono- or bicyclic heterocyclyl, optionally substituted mono- or bicyclic aryl, optionally substituted mono- or bicyclic heteroaryl,
   wherein said alkyl, alkenyl, alkynyl, cycloalkyl, heterocyclyl, aryl, or heteroaryl moiety is optionally substituted with from 1 to 3 substituents independently selected from the group consisting of halo, NO₂, CF₃, CN, OR²⁰, SR²⁰, N(R²⁰)₂, S(O)R²², SO₂R²², SO₂N(R²⁰)₂, S(O)₃R²⁰, P(O)(OR²⁰)₂, SO₂NR²⁰COR²², SO₂NR²⁰CO₂R²², SO₂NR²⁰ CON(R²⁰)₂, NR²⁰COR²², NR²⁰CO₂R²², NR²⁰CON(R²⁰)₂, NR²⁰C(NR²⁰)NHR²³, COR²⁰, CO₂R²⁰, CON(R²⁰)₂, CONR²⁰SO₂R²², NR²⁰SO₂R²², SO₂NR²⁰CO₂R²², OCONR²⁰SO₂R²², OC(O)R²⁰, C(O)OCH₂OC(O)R²⁰, OCON(R²⁰)₂, wherein R²⁰ and R²² are independently selected from the group consisting of hydrogen, C₁₋₁₅ alkyl, C₂₋₁₅ alkenyl, C₂₋₁₅ alkynyl, heterocyclyl, aryl, and heteroaryl;
X¹ is selected from: —O—C(O)—, —C(O)—O—, —NR′—C(O)—, —C(O)—NR′—, —O—C(O)—NR′—, —NR′—, —O—, —S—, —NR′—S(O)₂—, or —S(O)₂—NR′—, wherein R′ is hydrogen or C₁₋₆ lower alkyl.
L¹ is a covalent bond or -Lk-Y—, wherein Lk is optionally substituted linear or branched C₁₋₄ alkylene and Y is selected from a covalent bond, —O—, —S—, or —NR″—, wherein R″ is hydrogen or C₁₋₆ lower alkyl;
L² is a covalent bond or -Lk′-Y′—, wherein Lk′ is optionally substituted linear or branched C₁₋₄ alkylene and Y′ is selected from a covalent bond, —O—, —S—, or —NR″—, wherein R″ is hydrogen or C₁₋₆ lower alkyl;
W1 is —O— or —S—; and
the R²L²-NH— is bonded to the 6 or 7 position indicated in Formula I.

In some embodiments of the invention R¹ and R² are optionally substituted with from 1 to 3 substituents independently selected from the group consisting of alkyl, heterocyclyl, aryl, heteroaryl, halo, NO₂, CF₃, CN, OR²⁰, SR²⁰, N(R²⁰)₂, S(O)R²², SO₂R²², SO₂N(R²⁰)₂, S(O)₃R²⁰, P(O)(OR²⁰)₂, SO₂NR²⁰COR²², SO₂NR²⁰CO₂R²², SO₂NR²⁰CON(R²⁰)₂, NR²⁰R²², NR²⁰CO₂R²², NR²⁰CON(R²⁰)₂, NR²⁰C(NR²⁰)NHR²³, COR²⁰, CO₂R²⁰, CON(R²⁰)₂, CONR²⁰SO₂R²², NR²⁰SO₂R²², SO₂NR²⁰CO₂R²², OCONR²⁰SO₂R²², OC(O)R²⁰, C(O)OCH₂OC(O)R²⁰, and OCON(R²⁰)₂, and in some cases each optional alkyl, heteroaryl, aryl, and heterocyclyl substituent is further optionally substituted with halo, NO₂, alkyl, CF₃, amino, mono- or di-alkylamino, alkyl or aryl or heteroaryl amide, NR²⁰COR²², NR²⁰SO₂R²², COR²⁰, CO₂R²⁰, CON(R²⁰)₂, NR²⁰CON(R²⁰)₂, OC(O)R²⁰, OC(O)N(R²⁰)₂, S(O)₃R²⁰, P(O)(OR²⁰)₂, SR²⁰, S(O)R²², SO₂R²², SO₂N(R²⁰)₂, CN, or OR²⁰.

In certain embodiments of the invention R¹ and R² are optionally substituted with from 1 to 3 substituents independently selected from the group consisting of alkyl, heterocyclyl, aryl, heteroaryl, halo, NO₂, CF₃, CN, OR²⁰, SR²⁰, N(R²⁰)₂, S(O)R²², SO₂R²², SO₂N(R²⁰)₂, NR²⁰COR²², NR²⁰CO₂R²², NR²⁰CON(R²⁰)₂, COR²⁰, CO₂R²⁰, CON(R²⁰)₂, NR²⁰SO₂R²², and OC(O)R²⁰, and in some cases each optional alkyl, heteroaryl, aryl, and heterocyclyl substituent is further optionally substituted with halo, NO₂, alkyl, CF₃, amino, mono- or di-alkylamino, alkyl or aryl or heteroaryl amide, NR²⁰COR²², NR²⁰SO₂R²², COR²⁰, CO₂R²⁰, CON(R²⁰)₂, SR²⁰, S(O)R²², SO₂R²², SO₂N(R²⁰)₂, CN, or OR²⁰.

In typical embodiments R²⁰ and R²² are independently selected from the group consisting of hydrogen, C₁₋₁₅ alkyl, C₂₋₁₅ alkenyl, C₂₋₁₅ alkynyl, heterocyclyl, aryl, and heteroaryl, wherein the alkyl, alkenyl, alkynyl, heterocyclyl, aryl, and heteroaryl moieties are optionally substituted with from 1 to 3 substituents independently selected from halo, alkyl, mono- or dialkylamino, alkyl or aryl or heteroaryl amide, CN, C₁₋₆ alkyl-O—, CF₃, aryl, and heteroaryl.

In typical embodiments, the R¹ group is hydrogen, optionally substituted C₁₋₆ lower alkyl (e.g. methyl, ethyl, n-propyl, iso-propyl, n-butyl, iso-butyl, t-butyl, n-hexyl, trifluoromethyl, hydroxymethyl, hydroxyethyl, and the like), optionally substituted C₁₋₆ alkoxy (e.g. methoxy, ethoxy, n-propoxy, iso-propoxy, n-butoxy, tert-butoxy, sec-butoxy, n-pentoxy, n-hexoxy, 1,2-dimethylbutoxy, trifluoromethoxy, and the like), or optionally substituted phenyl (such as phenyl optionally substituted at the 2, 3, 4, and/or 5 position(s) of the phenyl ring with 1 to 3 substituents selected from the group consisting of halogen, methyl, ethyl, n-propyl, isopropyl, CF₃, —OCF₃, and —OCH₃).

In typical embodiments, the R² group is C₁₋₂₀ alkyl (e.g. methyl, ethyl, propyl, butyl, pentyl, hexyl, heptyl, or octyl) optionally substituted with 1, 2, or 3 substituents selected from the group consisting of hydroxy, halogen, NO₂, C₁₋₆ alkyl, C₁₋₆ alkyl-O—, CF₃, amino, mono- or di-alkylamino. In other typical embodiments, the R² group is optionally substituted aryl, such as a phenyl optionally substituted at the 2, 3, 4, or 5 position of the phenyl ring with 1, 2, or 3 substituents selected from the group consisting of halogen, CF₃, —OCF₃, —OCH₃, C₁₋₆ lower alkyl, C₁₋₆ alkoxy, C₁₋₆ alkylthio, aryl, or heteroaryl; in such embodiments the C₁₋₆ lower alkyl, C₁₋₆ alkoxy, C₁₋₆ alkylthio, aryl, or heteroaryl substituent(s) on the phenyl may themselves be optionally substituted with 1, 2, or 3 substituents selected from the group consisting of halogen, CF₃, —OCF₃, and —OCH₃.

In some embodiments, the R² group is optionally substituted phenyl, optionally substituted mono- or bicyclic heterocyclyl (e.g. pyridyl, furyl, indolizinyl, benzothiazolyl, benzothienyl, [1,2,4]oxadiazolyl, [1,3,4]oxadiazolyl, [1,2,4]thiadiazolyl, [1,3,4]thiadiazolyl, pyrrolyl, imidazolyl, pyrazolyl, pyridyl, pyrazyl, pyrimidinyl, pyridazinyl, indolizinyl, isoindolyl, indolyl, indazolyl, purinyl, quinolizinyl, isoquinolinyl, quinolinyl, phthalazinyl, naphthylpyridinyl, quinoxalyl, quinazolyl, cinnolyl, pteridyl, carbazolyl, phenanthridinyl, acridinyl, phenanthrolinyl, isothiazolyl, phenazinyl, isoxazolyl, phenoxazinyl, phenothiazinyl, imidazolidinyl, imidazolinyl), optionally substituted mono- or bicyclic aryl (e.g. phenyl, naphthyl and the like).

In typical embodiments, R³ and R⁴ are independently selected from hydrogen, optionally substituted C₁₋₆ alkyl, optionally substituted C₁₋₆ alkoxyl, fluoro, trifluoromethyl, 2,2,2-trifluoroethyl, trifluoromethoxyl, ethyloxycarbonyl, carboxyl, phenethyl, optionally substituted pyridyl, optionally substituted phenyl (such as, but not limited to, methoxyphenyl, methylthiophenyl, methoxyethoxyphenyl, propylphenyl, acetamidophenyl, methylsulfonylphenyl, dichlorophenyl, or chlorophenyl). In some embodiments R³ and R⁴ are independently selected from the group consisting of hydrogen, optionally substituted C₁₋₆ alkyl, optionally substituted five or six membered monocyclic heterocyclyl, optionally substituted phenyl, optionally substituted five or six membered monocyclic heteroaryl. In certain embodiments R³ and R⁴ are independently selected from the group consisting of hydrogen, methyl, ethyl, propyl, trifluoromethyl, perfluoroethyl, pyridyl, and optionally substituted phenyl. In some embodiments R³ and R⁴ are independently selected from the group consisting of hydrogen and phenyl optionally substituted with 1, 2, or 3 substituents selected from the group consisting of methyl, methoxy, ethyl, ethoxy, propyl, propoxy, trifluoromethyl, trifluoromethoxyl, perfluoroethyl, pyridyl, or C₁₋₆ alkyl.

In certain embodiments the L¹ group is a covalent bond or -Lk-Y—, wherein Lk is optionally substituted linear or branched C₁₋₄ alkylene and Y is selected from a covalent bond, —O—, —S—, or —NR″—, wherein R″ is hydrogen or C₁₋₆ lower alkyl. In some embodiments the L¹ group may be a C₁₋₄ alkylene optionally substituted with one or two substituents selected from hydroxyl, lower alkyl, lower alkoxy, halogen, CF₃, and —OCF₃. Typical L¹ groups are covalent bond, optionally substituted C₁₋₄ alkylene-Y—, optionally substituted C₂₋₃ alkylene-Y—, methylene-Y—, —CH₂CH₂—Y—, —CH₂CH₂CH₂—Y—; —CH(CH₃)CH₂—Y—, —CH₂CH₂CH₂CH₂—Y—, —C(CH₃)₂CH₂—Y— or —CH(CH₃)CH₂CH₂—Y—, wherein Y is selected from a covalent bond, —O—, —S—, or —NR″—, wherein R″ is hydrogen or C₁₋₆ lower alkyl. Typically, Y is selected from covalent bond or —O—. In typical embodiments, L¹ is oriented so that Y is directly connected to the X¹ group; in other embodiments, it is the Lk that is directly connected to the X¹ group.

In certain embodiments the L² group is a covalent bond or —Lk′—Y′—, wherein Lk′ is optionally substituted linear or branched C₁₋₄ alkylene and Y′ is selected from a covalent bond, —O—, —S—, or —NR″—, wherein R″ is hydrogen or C₁₋₆ lower alkyl. In some embodiments the L² group may be a C₁₋₄ alkylene optionally substituted with one or two substituents selected from hydroxyl, lower alkyl, lower alkoxy, halogen, CF₃, and —OCF₃. Typical L² groups are covalent bond, optionally substituted C₁₋₄ alkylene-Y′—, optionally substituted C₂₋₃ alkylene-Y′—, methylene-Y′—, —CH₂CH₂—Y′—, —CH₂CH₂CH₂—Y′—; —CH(CH₃)CH₂—Y′—, —CH₂CH₂CH₂CH₂—Y′—, —C(CH₃)₂CH₂—Y′— or —CH(CH₃)CH₂CH₂—Y′—, wherein Y′ is selected from a covalent bond, —O—, —S—, or —NR″—, wherein R″ is hydrogen or C₁₋₆ lower alkyl. Typically, Y′ is selected from covalent bond or —O—. Typical L² groups are covalent bond, C₂₋₃ alkylene, methylene, —CH₂CH₂—, —CH₂CH₂CH₂—; —CH(CH₃)CH₂—. In typical embodiments, L² is oriented so that Y′ is directly connected to the R²group; in other embodiments, it is the Lk′ that is directly connected to the R² group.

Typical L¹ groups are covalent bond, C₂₋₃ alkylene, methylene, —CH₂CH₂—, —CH₂CH₂CH₂—; —CH(CH₃)CH₂—, —CH₂CH₂CH₂CH₂—, —C(CH₃)₂CH₂— or —CH(CH₃)CH₂CH₂—. In some embodiments the L¹ group may be a C₁₋₄ alkylene substituted with one or two substituents selected from hydroxyl, lower alkyl, lower alkoxy, halogen, CF₃, and —OCF₃.

Typical L² groups are covalent bond, C₂₋₃ alkylene, methylene, —CH₂CH₂—, —CH₂CH₂CH₂—; —CH(CH₃)CH₂—, —CH₂CH₂CH₂CH₂—, —C(CH₃)₂CH₂— or —CH(CH₃)CH₂CH₂—. In some embodiments the L2 group may be a C₁₋₄ alkylene substituted with one or two substituents selected from hydroxyl, lower alkyl, lower alkoxy, halogen, CF₃, and —OCF₃.

In typical embodiments X¹ is a moiety selected from: —O—C(O)—, —NR′—C(O)—, —C(O)—NR′—, —O—C(O)—NR′—, —NR′—, —O—, —S—, —NR′—S(O)₂—, or —S(O)₂—NR′—, wherein R′ is hydrogen or C₁₋₆ lower alkyl. In certain embodiments the X¹group is selected from —O—C(O)—, —NR′—C(O)—, —C(O)—NR′—, —NR′—, or —O—, wherein R′ is hydrogen or C₁₋₆ lower alkyl (e.g. methyl, ethyl, propyl, butyl, pentyl, or hexyl). In certain embodiments the X¹ group is selected from —NR′—C(O)— or —C(O)—NR′ . . . wherein R′ is hydrogen or C₁₋₆ lower alkyl (e.g. methyl, ethyl, propyl, butyl, pentyl, or hexyl). In typical embodiments, the X¹ group is oriented such that the first portion written of the X¹ group (as written herein, writing from left to right in the normal manner) is directly attached to L¹. Thus, the —NR′—C(O)— has the nitrogen directly connected to L¹, and the —C(O)—NR′— has the carbon directly connected to L¹.

In typical embodiments W1 is —O—; in other embodiments W1 is —S—.

In some embodiments, the R²—L2-NH— moiety is attached to the 7 position of the benzo[b][1,4]oxazin-3-one and the compound has the stricture of Formula Ia:

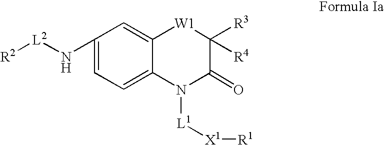

In other embodiments, the R²—L2-NH— moiety is attached to the 6 position of the benzo[b][1,4]oxazin-3-one and the compound has the structure of Formula Ib:

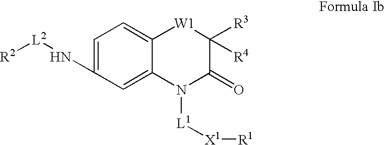

In yet another aspect of the invention, pharmaceutical formulations are provided, comprising a therapeutically effective amount of an SCD inhibitory compound of Formula I, and at least one pharmaceutically acceptable carrier. The formulation is typically for oral administration, but in some embodiments may be provided for administration via other routes.

In a third embodiment of the invention, methods of using the compounds of Formula I in the treatment of a disease or condition in a mammal that can be treated with an SCD inhibitory compound are provided. The method comprises administering to a mammal in need thereof a therapeutically effective dose of a compound of Formula I. Such diseases include, but are not limited to, cardiovascular diseases (including, but not limited to, coronary artery disease, atherosclerosis, heart disease, hypertension, and peripheral vascular disease), cancer, cerebrovascular diseases (including, but not limited to, stroke, ischemic stroke and transient ischemic attack (TIA), and ischemic retinopathy), dyslipidemia, obesity, diabetes, insulin resistance, decreased glucose tolerance, non-insulin-dependent diabetes mellitus, Type II diabetes, Type I diabetes, and other diabetic complications.

At present, the compounds for use in the invention include, but are not limited to:

3-(6-(3,4-dichlorobenzylamino)-3-oxo-2H-benzo[b][1,4]oxazin-4(3H)-yl)propanamide;

N-(2-(6-(benzylamino)-3-oxo-2H-benzo[b][1,4]oxazin-4(3H)-yl)ethyl)acetamide;

N-(2-(6-(3,4-dichlorobenzylamino)-3-oxo-2H-benzo[b][1,4]oxazin-4(3H)-yl)ethyl)acetamide;

6-(3,4-dichlorobenzylamino)-4-(2-phenoxyethyl)-2H-benzo[b][1,4]oxazin-3(4H)-one;

N-(2-(6-(4-chloro-3-(trifluoromethyl)benzylamino)-3-oxo-2H-benzo[b][1,4]oxazin-4(3H)-yl)ethyl)acetamide;

N-(2-(6-(4-fluoro-3-(trifluoromethyl)benzylamino)-3-oxo-2H-benzo[b][1,4]oxazin-4(3H)yl)ethyl)acetamide hydrochloride;

N-(2-(6-(3,4-dichlorobenzylamino)-3-oxo-2H-benzo[b][1,4]oxazin-4(3H)-yl)ethyl)benzamide;

N-(2-(6-(3,4-dichlorobenzylamino)-3-oxo-2H-benzo[b][1,4]oxazin-4(3H)-yl)ethyl)-2-hydroxyacetamide hydrochloride;

(±)-N-(2-(6-(3,4-dichlorobenzylamino)-2-methyl-3-oxo-2H-benzo[b][1,4]oxazin-4(3H)-yl)ethyl)acetamide;

(±)-N-(2-(6-(4-chloro-3-(trifluoromethyl)benzylamino)-2-methyl-3-oxo-2H-benzo[b][1,4]oxazin-4(3H)-yl)ethyl)acetamide;

(±)-N-(2-(6-(3,4-dichlorobenzylamino)-2-methyl-3-oxo-2H-benzo[b][1,4]oxazin-4(3H)-yl)ethyl)-2-hydroxyacetamide;

(±)-N-(2-(6-(4-chloro-3-(trifluoromethyl)benzylamino)-2-methyl-3-oxo-2H-benzo[b][1,4]oxazin-4(3H)-yl)ethyl)-2-hydroxyacetamide;

N-(2-(6-(3,4-dichlorobenzylamino)-2,2-dimethyl-3-oxo-2H-benzo[b][1,4]oxazin-4(3H)-yl)ethyl)acetamide;

N-(2-(6-(3,4-dichlorobenzylamino)-2,2-dimethyl-3-oxo-2H-benzo[b][1,4]oxazin-4(3H)-yl)ethyl)-2-hydroxyacetamide;

N-(2-(7-(3,4-dichlorobenzylamino)-3-oxo-2H-benzo[b][1,4]oxazin-4(3H)-yl)ethyl)acetamide; and

N-(2-(7-(3,4-dichlorobenzylamino)-3-oxo-2H-benzo[b][1,4]oxazin-4(3H)-yl)ethyl)-2-hydroxyacetamide.

### DETAILED DESCRIPTION OF THE INVENTION

### Definitions and General Parameters

As used in the present specification, the following words and phrases are generally intended to have the meanings as set forth below, except to the extent that the context in which they are used indicates otherwise.

The term “alkyl” refers to a monoradical branched or unbranched saturated hydrocarbon chain having 1, 2, 3, 4, 5, 6, 7, 8, 9, 10, 11, 12, 13, 14, 15, 16, 17, 18, 19 or 20 carbon atoms. This term is exemplified by groups such as methyl, ethyl, n-propyl, iso-propyl, n-butyl, iso-butyl, t-butyl, n-hexyl, n-decyl, tetradecyl, and the like.

The term “substituted alkyl” refers to:
1) an alkyl group as defined above, having 1, 2, 3, 4 or 5 substituents, typically 1 to 3 substituents, selected from the group consisting of alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thio, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2; or
2) an alkyl group as defined above that is interrupted by 1-10 atoms independently chosen from oxygen, sulfur and NRₐ—, where Rₐ is chosen from hydrogen, alkyl, cycloalkyl, alkenyl, cycloalkenyl, alkynyl, aryl, heteroaryl and heterocyclyl. All substituents may be optionally further substituted by alkyl, alkoxy, halogen, CF₃, amino, substituted amino, cyano, or —S(O)ₙR, in which R is alkyl, aryl, or heteroaryl and n is 0, 1 or 2; or
3) an alkyl group as defined above that has both 1, 2, 3, 4 or 5 substituents as defined above and is also interrupted by 1-10 atoms as defined above.

The term “lower alkyl” refers to a monoradical branched or unbranched saturated hydrocarbon chain having 1, 2, 3, 4, 5, or 6 carbon atoms. This term is exemplified by groups such as methyl, ethyl, n-propyl, iso-propyl, n-butyl, iso-butyl, t-butyl, n-hexyl, and the like.

The term “substituted lower alkyl” refers to lower alkyl as defined above having 1 to 5 substituents, typically 1, 2, or 3 substituents, as defined for substituted alkyl, or a lower alkyl group as defined above that is interrupted by 1, 2, 3, 4, or 5 atoms as defined for substituted alkyl, or a lower alkyl group as defined above that has both 1, 2, 3, 4 or 5 substituents as defined above and is also interrupted by 1, 2, 3, 4, or 5 atoms as defined above.

The term “alkylene” refers to a diradical of a branched or unbranched saturated hydrocarbon chain, having 1, 2, 3, 4, 5, 6, 7, 8, 9, 10, 11, 12, 13, 14, 15, 16, 17, 18, 19 or 20 carbon atoms, typically 1-10 carbon atoms, more typically 1, 2, 3, 4, 5 or 6 carbon atoms. This term is exemplified by groups such as methylene (—CH₂—), ethylene (—CH₂CH₂—), the propylene isomers (e.g., —CH₂CH₂CH₂— and —CH(CH₃)CH₂—) and the like.

The term “lower alkylene” refers to a diradical of a branched or unbranched saturated hydrocarbon chain, typically having from 1, 2, 3, 4, 5, or 6 carbon atoms.

The term “lower alkylene” refers to a diradical of a branched or unbranched saturated hydrocarbon chain, typically having from 1, 2, 3, 4, 5, or 6 carbon atoms.

The term “substituted alkylene” refers to:
(1) an alkylene group as defined above having 1, 2, 3, 4, or 5 substituents selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2; or
(2) an alkylene group as defined above that is interrupted by 1-20 atoms independently chosen from oxygen, sulfur and NRₐ—, where Rₐ is chosen from hydrogen, optionally substituted alkyl, cycloalkyl, cycloalkenyl, aryl, heteroaryl and heterocycyl, or groups selected from carbonyl, carboxyester, carboxyamide and sulfonyl; or
(3) an alkylene group as defined above that has both 1, 2, 3, 4 or 5 substituents as defined above and is also interrupted by 1-20 atoms as defined above. Examples of substituted alkylenes are chloromethylene (—CH(Cl)—), aminoethylene (—CH(NH₂)CH₂—), methylaminoethylene (—CH(NHMe)CH₂—), 2-carboxypropylene isomers (—CH₂CH(CO₂H)CH₂—), ethoxyethyl (—CH₂CH₂O—CH₂CH₂—), ethylmethylaminoethyl (—CH₂CH₂N(CH₃)CH₂CH₂—), 1-ethoxy-2-(2-ethoxy-ethoxy)ethane (—CH₂CH₂O—CH₂CH₂—OCH₂CH₂—OCH₂CH₂—), and the like.

The term “aralkyl” refers to an aryl group covalently linked to an alkylene group, where aryl and alkylene are defined herein. “Optionally substituted aralkyl” refers to an optionally substituted aryl group covalently linked to an optionally substituted alkylene group. Such aralkyl groups are exemplified by benzyl, phenylethyl, 3-(4-methoxyphenyl)propyl, and the like.

The term “alkoxy” refers to the group R—O—, where R is optionally substituted alkyl or optionally substituted cycloalkyl, or R is a group —Y-Z, in which Y is optionally substituted alkylene and Z is optionally substituted alkenyl, optionally substituted alkynyl; or optionally substituted cycloalkenyl, where alkyl, alkenyl, alkynyl, cycloalkyl and cycloalkenyl are as defined herein. Typical alkoxy groups are optionally substituted alkyl-O— and include, by way of example, methoxy, ethoxy, n-propoxy, iso-propoxy, n-butoxy, tert-butoxy, sec-butoxy, n-pentoxy, n-hexoxy, 1,2-dimethylbutoxy, trifluoromethoxy, and the like.

The term “alkylthio” refers to the group R—S—, where R is as defined for alkoxy.

The term “alkenyl” refers to a monoradical of a branched or unbranched unsaturated hydrocarbon group typically having from 2 to 20 carbon atoms, more typically 2 to 10 carbon atoms and even more typically 2 to 6 carbon atoms and having 1-6, typically 1, double bond (vinyl). Typical alkenyl groups include ethenyl or vinyl (—CH═CH₂), 1-propylene or allyl (—CH₂CH═CH₂), isopropylene (—C(CH₃)═CH₂), bicyclo[2.2.1]heptene, and the like. In the event that alkenyl is attached to nitrogen, the double bond cannot be alpha to the nitrogen.

The term “lower alkenyl” refers to alkenyl as defined above having from 2 to 6 carbon atoms.

The term “substituted alkenyl” refers to an alkenyl group as defined above having 1, 2, 3, 4 or 5 substituents, typically 1, 2, or 3 substituents, selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroalyl. Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or2.

The term “alkynyl” refers to a monoradical of an unsaturated hydrocarbon, typically having from 2 to 20 carbon atoms, more typically 2 to 10 carbon atoms and even more typically 2 to 6 carbon atoms and having at least 1 and typically from 1-6 sites of acetylene (triple bond) unsaturation. Typical alkynyl groups include ethynyl, (—C≡CH), propargyl (or prop-1-yn-3-yl, —CH₂C≡CH), and the like. In the event that alkynyl is attached to nitrogen, the triple bond cannot be alpha to the nitrogen.

The term “substituted alkynyl” refers to an alkynyl group as defined above having 1, 2, 3, 4 or 5 substituents, and typically 1, 2, or 3 substituents, selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2.

The term “aminocarbonyl” refers to the group —C(O)NRR where each R is independently hydrogen, alkyl, aryl, heteroaryl, heterocyclyl or where both R groups are joined to form a heterocyclic group (e.g., morpholino). Unless otherwise constrained by the definition, all substituents may optionally be further substituted by 1-3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2.

The term “acylamino” refers to the group —NRC(O)R where each R is independently hydrogen, alkyl, aryl, heteroaryl, or heterocyclyl. Unless otherwise constrained by the definition, all substituents may optionally be further substituted by 1-3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2.

The term “acyloxy” refers to the groups —O(O)C-alkyl, —O(O)C-cycloalkyl, —O(O)C-aryl, —O(O)C-heteroaryl, and —O(O)C-heterocyclyl. Unless otherwise constrained by the definition, all substituents may be optionally further substituted by alkyl, carboxy, carboxyalkyl, aminocarbonyl, hydroxy, alkoxy, halogen, CF₃, amino, substituted amino, cyano, or —S(O)ₙR, where R is alkyl, aryl, or heteroaryl and n is 0, 1 or 2.

The term “aryl” refers to an aromatic carbocyclic group of 6 to 20 carbon atoms having a single ring (e.g., phenyl) or multiple rings (e.g., biphenyl), or multiple condensed (fused) rings (e.g., naphthyl or anthryl). Typical aryls include phenyl, naphthyl and the like.

The term “arylene” refers to a diradical of an aryl group as defined above. This term is exemplified by groups such as 1,4-phenylene, 1,3-phenylene, 1,2-phenylene, 1,4′-biphenylene, and the like.

Unless otherwise constrained by the definition for the aryl or arylene substituent, such aryl or arylene groups can optionally be substituted with from 1 to 5 substituents, typically 1 to 3 substituents, selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1-3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2.

The term “aryloxy” refers to the group aryl-O— wherein the aryl group is as defined above, and includes optionally substituted aryl groups as also defined above. The term “arylthio” refers to the group R—S—, where R is as defined for aryl.

The term “amino” refers to the group —NH₂.

The term “substituted amino” refers to the group —NRR where each R is independently selected from the group consisting of hydrogen, alkyl, cycloalkyl, carboxyalkyl (for example, benzyloxycarbonyl), aryl, heteroaryl and heterocyclyl provided that both R groups are not hydrogen, or a group —Y-Z, in which Y is optionally substituted alkylene and Z is alkenyl, cycloalkenyl, or alkynyl, Unless otherwise constrained by the definition, all substituents may optionally be further substituted by 1-3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2.

The term “carboxyalkyl” refers to the groups —C(O)O-alkyl or —C(O)O-cycloalkyl, where alkyl and cycloalkyl, are as defined herein, and may be optionally further substituted by alkyl, alkenyl, alkynyl, alkoxy, halogen, CF₃, amino, substituted amino, cyano, or —S(O)ₙR, in which R is alkyl, aryl, or heteroaryl and n is 0, 1 or 2.

The term “cycloalkyl” refers to carbocyclic groups of from 3 to 20 carbon atoms having a single cyclic ring or multiple condensed rings. Such cycloalkyl groups include, by way of example, single ring structures such as cyclopropyl, cyclobutyl, cyclopentyl, cyclooctyl, and the like, or multiple ring structures such as adamantanyl, bicyclo[2.2.1]heptane, 1,3,3-trimethylbicyclo[2.2.1]hept-2-yl, (2,3,3-trimethylbicyclo[2.2.1]hept-2-yl), or carbocyclic groups to which is fused an aryl group, for example indane, and the like.

The term “substituted cycloalkyl” refers to cycloalkyl groups having 1, 2, 3, 4 or 5 substituents, and typically 1, 2, or 3 substituents, selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2.

The term “halogen” or “halo” refers to fluoro, bromo, chloro, and iodo.

The term “acyl” denotes a group —C(O)R, in which R is hydrogen, optionally substituted alkyl, optionally substituted cycloalkyl, optionally substituted heterocyclyl, optionally substituted aryl, and optionally substituted heteroaryl.

The term “heteroaryl” refers to a radical derived from an aromatic cyclic group (i.e., fully unsaturated) having 1, 2, 3, 4, 5, 6, 7, 8, 9, 10, 11, 12, 13, 14, or 15 carbon atoms and 1, 2, 3 or 4 heteroatoms selected from oxygen, nitrogen and sulfur within at least one ring. Such heteroaryl groups can have a single ring (e.g., pyridyl or furyl) or multiple condensed rings (e.g., indolizinyl, benzothiazolyl, or benzothienyl). Examples of heteroaryls include, but are not limited to, [1,2,4]oxadiazole, [1,3,4]oxadiazole, [1,2,4]thiadiazole, [1,3,4]thiadiazole, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, and the like as well as N-oxide and N-alkoxy derivatives of nitrogen containing heteroaryl compounds, for example pyridine-N-oxide derivatives.

The term “heteroarylene” refers to a diradical of a heteroaryl group as defined above. This term is exemplified by groups such as 2,5-imidazolene, 3,5-[1,2,4]oxadiazolene, 2,4-oxazolene, 1,4-pyrazolene, and the like. For example, 1,4-pyrazolene is:

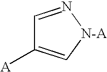

where A represents the point of attachment.

Unless otherwise constrained by the definition for the heteroaryl or heteroarylene substituent, such heteroaryl or heterarylene groups can be optionally substituted with 1 to 5 substituents, typically 1 to 3 substituents, selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1-3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2.

The term “heteroaralkyl” refers to a heteroaryl group covalently linked to an alkylene group, where heteroaryl and alkylene are defined herein. “Optionally substituted heteroaralkyl” refers to an optionally substituted heteroaryl group covalently linked to an optionally substituted alkylene group. Such heteroaralkyl groups are exemplified by 3-pyridylmethyl, quinolin-8-ylethyl, 4-methoxythiazol-2-ylpropyl, and the like.

The term “heteroaryloxy” refers to the group heteroaryl-O—.

The term “heterocyclyl” refers to a monoradical saturated or partially unsaturated group having a single ring or multiple condensed rings, having from 1 to 40 carbon atoms and from 1 to 10 hetero atoms, typically 1, 2, 3 or 4 heteroatoms, selected from nitrogen, sulfur, phosphorus, and/or oxygen within the ring. Heterocyclic groups can have a single ring or multiple condensed rings, and include tetrahydrofuranyl, morpholino, piperidinyl, piperazino, dihydropyridino, and the like.

Unless otherwise constrained by the definition for the heterocyclic substituent, such heterocyclic groups can be optionally substituted with 1, 2, 3, 4 or 5, typically 1, 2 or 3 substituents, selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1-3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2.

The term “thiol” refers to the group —SH.

The term “substituted alkylthio” refers to the group —S-substituted alkyl.

The term “heteroarylthio” refers to the group —S-heteroaryl wherein the heteroaryl group is as defined above including optionally substituted heteroaryl groups as also defined above.

The term “sulfoxide” refers to a group —S(O)R, in which R is alkyl, aryl, or heteroaryl. “Substituted sulfoxide” refers to a group —S(O)R, in which R is substituted alkyl, substituted aryl, or substituted heteroaryl, as defined herein.

The term “sulfone” refers to a group —S(O)₂R, in which R is alkyl, aryl, or heteroaryl. “Substituted sulfone” refers to a group —S(O)₂R, in which R is substituted alkyl, substituted aryl, or substituted heteroaryl, as defined herein.

The term “keto” refers to a group —C(O)—.

The term “thiocarbonyl” refers to a group —C(S)—.

The term “carboxy” refers to a group —C(O)—OH.

“Optional” or “optionally” means that the subsequently described event or circumstance may or may not occur, and that the description includes instances where said event or circumstance occurs and instances in which it does not.

The term “compound of Formula I” is intended to encompass the compounds of the invention as disclosed, and the pharmaceutically acceptable salts, pharmaceutically acceptable esters, prodrugs, hydrates and polymorphs of such compounds. Additionally, the compounds of the invention may possess one or more asymmetric centers, and can be produced as a racemic mixture or as individual enantiomers or diastereoisomers. The number of stereoisomers present in any given compound of Formula I depends upon the number of asymmetric centers present (there are 2ⁿ stereoisomers possible where n is the number of asymmetric centers). The individual stereoisomers may be obtained by resolving a racemic or non-racemic mixture of an intermediate at some appropriate stage of the synthesis, or by resolution of the compound of Formula I by conventional means. The individual stereoisomers (including individual enantiomers and diastereoisomers) as well as racemic and non-racemic mixtures of stereoisomers are encompassed within the scope of the present invention, all of which are intended to be depicted by the structures of this specification unless otherwise specifically indicated.

“Isomers” are different compounds that have the same molecular formula.

“Stereoisomers” are isomers that differ only in the way the atoms are arranged in space.

“Enantiomers” are a pair of stereoisomers that are non-superimposable mirror images of each other. A 1:1 mixture of a pair of enantiomers is a “racemic” mixture. The term “(±)” is used to designate a racemic mixture where appropriate.

“Diastereoisomers” are stereoisomers that have at least two asymmetric atoms, but which are not mirror-images of each other.

The absolute stereochemistry is specified according to the Cahn-Ingold-Prelog R—S system. When the compound is a pure enantiomer the stereochemistry at each chiral carbon may be specified by either R or S. Resolved compounds whose absolute configuration is unknown are designated (+) or (−) depending on the direction (dextro- or laevorotary) which they rotate the plane of polarized light at the wavelength of the sodium D line.

“Parenteral administration” is the systemic delivery of the therapeutic agent via injection to the patient.

The term “therapeutically effective amount” refers to that amount of a compound of Formula I that is sufficient to effect treatment, as defined below, when administered to a mammal in need of such treatment. The therapeutically effective amount will vary depending upon the specific activity of the therapeutic agent being used, and the age, physical condition, existence of other disease states, and nutritional status of the patient. Additionally, other medication the patient may be receiving will effect the determination of the therapeutically effective amount of the therapeutic agent to administer.

The term “treatment” or “treating” means any treatment of a disease in a mammal, including:
(i) preventing the disease, that is, causing the clinical symptoms of the disease not to develop;
(ii) inhibiting the disease, that is, arresting the development of clinical symptoms; and/or
(iii) relieving the disease, that is, causing the regression of clinical symptoms.

In many cases, the compounds of this invention are capable of forming acid and/or base salts by virtue of the presence of amino and/or carboxyl groups or groups similar thereto. The term “pharmaceutically acceptable salt” refers to salts that retain the biological effectiveness and properties of the compounds of Formula I and which are not biologically or otherwise undesirable. Pharmaceutically acceptable base addition salts can be prepared from inorganic and organic bases. Salts derived from inorganic bases, include by way of example only, sodium, potassium, lithium, ammonium, calcium and magnesium salts. Salts derived from organic bases include, but are not limited to, salts of primary, secondary and tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, disubstituted cycloalkyl amine, trisubstituted cycloalkyl amines, cycloalkenyl amines, di(cycloalkenyl) amines, tri(cycloalkenyl) amines, substituted cycloalkenyl amines, disubstituted cycloalkenyl amine, trisubstituted cycloalkenyl amines, aryl amines, diaryl amines, triaryl amines, heteroaryl amines, d heteroaryl amines, triheteroaryl amines, heterocyclic amines, diheterocyclic amines, triheterocyclic amines, mixed di- and tri-amines where at least two of the substituents on the amine are different and are selected from the group consisting of alkyl, substituted alkyl, alkenyl, substituted alkenyl, cycloalkyl, substituted cycloalkyl, cycloalkenyl, substituted cycloalkenyl, aryl, heteroaryl, heterocyclic, and the like. Also included are amines where the two or three substituents, together with the amino nitrogen, form a heterocyclic or heteroaryl group.

Specific examples of suitable amines include, by way of example only, isopropylamine, trimethyl amine, diethyl amine, tri(iso-propyl) amine, tri(n-propyl) amine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, morpholine, N-ethylpiperidine, and the like.

Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

### Nomenclature

Names of compounds of the present invention are provided using ChemDraw Ultra v. 10.0 (CambridgeSoft, Cambridge, Mass.). Some compounds or radicals may be named with common names, or systematic or non-systematic names. The naming of the compounds of the invention is illustrated with a representative compound of Formula I in which R¹ is methyl, R² is 4-chloro-3-(trifluoromethyl)phenyl, L1 is —CH₂CH₂—, L2 is methylene, X1 is —NH—C(O)—, and W1 is —O—.

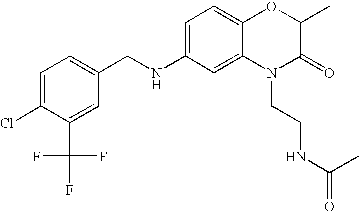

which is named:
(±)-N-(2-(6-(4-chloro-3-(trifluoromethylbenzylamino)-2-methyl-3-oxo-2H-benzo[b][1,4]oxazin-4(3H)-yl)ethyl)acetamide.

### Pharmaceutical Compositions

When selected as the SCD inhibitor, the compounds of Formula I are usually administered in the form of pharmaceutical compositions. This invention therefore provides pharmaceutical compositions that contain, as the active ingredient, one or more of the compounds of Formula I, or a pharmaceutically acceptable salt or ester thereof, and one or more pharmaceutically acceptable excipients, carriers, including inert solid diluents and fillers, diluents, including sterile aqueous solution and various organic solvents, solubilizers and adjuvants. The compounds of Formula I may be administered alone or in combination with other therapeutic agents. Such compositions are prepared in a manner well known in the pharmaceutical art (see, e.g., Remington's Pharmaceutical Sciences, Mace Publishing Co., Philadelphia, Pa. 17^{th} Ed. (1985) and “Modem Pharmaceutics”, Marcel Dekker, Inc. 3^{rd} Ed. (G.S. Banker & C.T. Rhodes, Eds.).

### Synthetic Reaction Parameters

The terms “solvent”, “inert organic solvent” or “inert solvent” mean a solvent inert under the conditions of the reaction being described in conjunction therewith (including, for example, benzene, toluene, acetonitrile, tetrahydrofuran (“THF”), dimethylformamide (“DMF”), chloroform, methylene chloride (or dichloromethane), diethyl ether, methanol, pyridine and the like). Unless specified to the contrary, the solvents used in the reactions of the present invention are inert organic solvents, and the reactions are carried out under an inert gas, typically nitrogen.

The term “q.s.” means adding a quantity sufficient to achieve a stated function, e.g., to bring a solution to the desired volume (i.e., 100%).

### Synthesis of the Compounds of Formula I

The compounds of Formula I are typically prepared by first synthesizing a precursor core compound and then sequentially adding the L²R² and L¹X¹R¹ moieties. The general method of preparing the compounds of Formula I is shown in Reaction Scheme I.

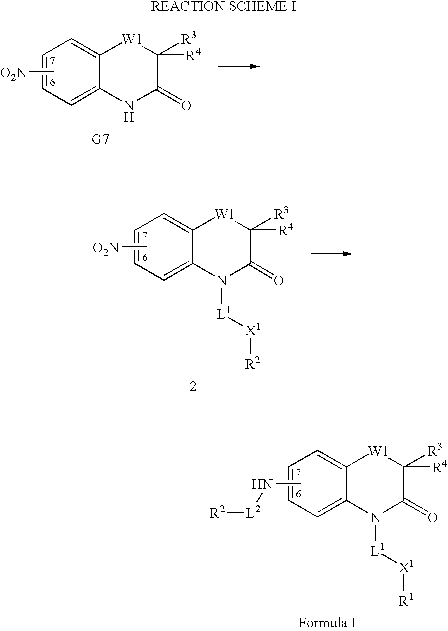

### Preparation of Precursor Core Compound of Formula G7

Compounds of Formula I are prepared by first synthesizing a precursor core compound, such as compound G7, by reaction of a suitable aniline G5 with a dihalogen compound G6 leading to cyclization and formation of the compound G7. This reaction is carried our in suitable solvent (e.g. acetone) in the presence of base, such as triethylamine (preferred), pyridine, diisopropylethylamine, or potassium carbonate. See Reaction Scheme Ia below.

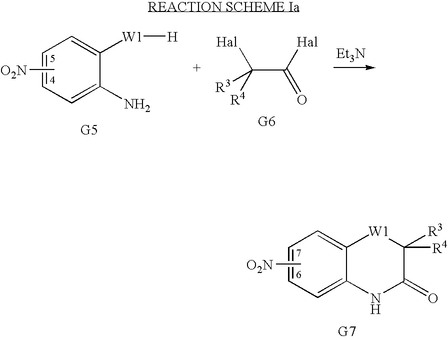

### Addition of L¹X¹R¹ Group

The L¹X¹R¹ group may be added to the precursor core by reaction of a halogenated L¹X¹R¹ derivative such as compound G10 with the compound G7 The reaction is typically performed in organic solvent, such as DMF or DMSO, in the presence of a base such sodium hydroxide or the like, to produce the intermediate compound G11. The reaction is illustrated below in Reaction Scheme Ia below.

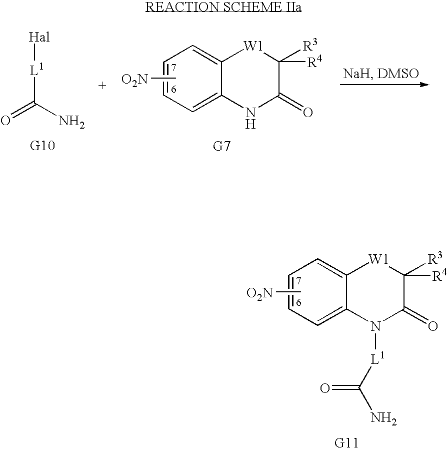

### Alternative Addition of L¹X¹R¹ Group

The L¹X¹R¹ group may be also be added to the precursor core in a two step process by first reacting a halogenated L¹-isoindoline-1,3-dione derivative such as compound G4 with the compound G7 to produce the coupling product G8 and then removing the isoindolinedione moiety to arrive at an amino intermediate G9.

As was the case the G10/G7 coupling described above, the reaction of compound G4 with the compound G7 is typically performed in organic solvent, such as DMF or DMSO, in the presence of a base such sodium hydroxide or the like. The coupling product G8 is converted into free amine G9 by heating with methylamine or hydrazine (preferred) in a suitable solvent (e.g. ethanol). If desired, the resulting free base may be converted into HCl salt using any suitable source of hydrochloric acid (e.g. 4N HCl in dioxane). The reaction is illustrated below in Reaction Scheme IIb below.

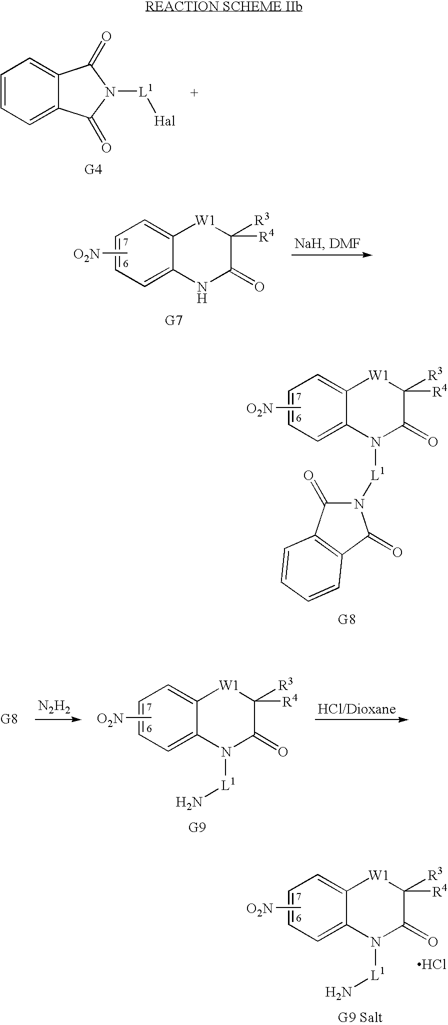

### Preparation of Compound of Formula G4

Precursor compounds G4 are commercially available where Hal=Br. Compounds where Hal=I may be prepared by reacting phthalic anhydride G1 with aminoalcohol of Formula G2 to produce alcohols of Formula G3. This reaction may require heating and can be carried out in any suitable organic solvent, or, in one typical method, neat (without solvent). Compounds of Formula G3, are then converted to iodo-derivatives (G4, Hal=I). This reaction can be carried out by a number of methods including, but not limited to, HI, I₂ in the presence of phosphorus, Ph₃P/N-iodosuccinimide. One method includes using Ph₃P, I₂, and imidazole in dichloromethane as shown in Reaction Scheme IIc below.

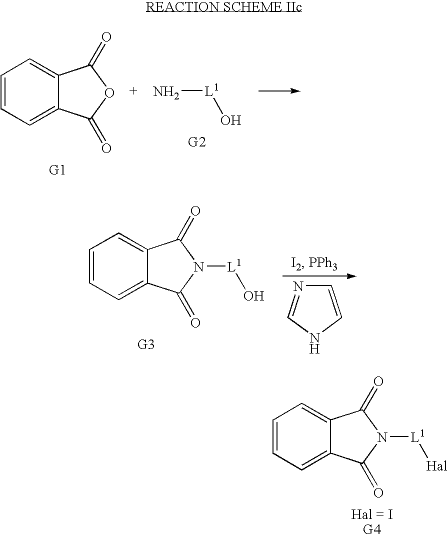

### Addition of L²R² Group

A typical method for coupling the L2R2 moiety to the compound core is illustrated in Reaction Scheme III below, this reaction is illustrated.

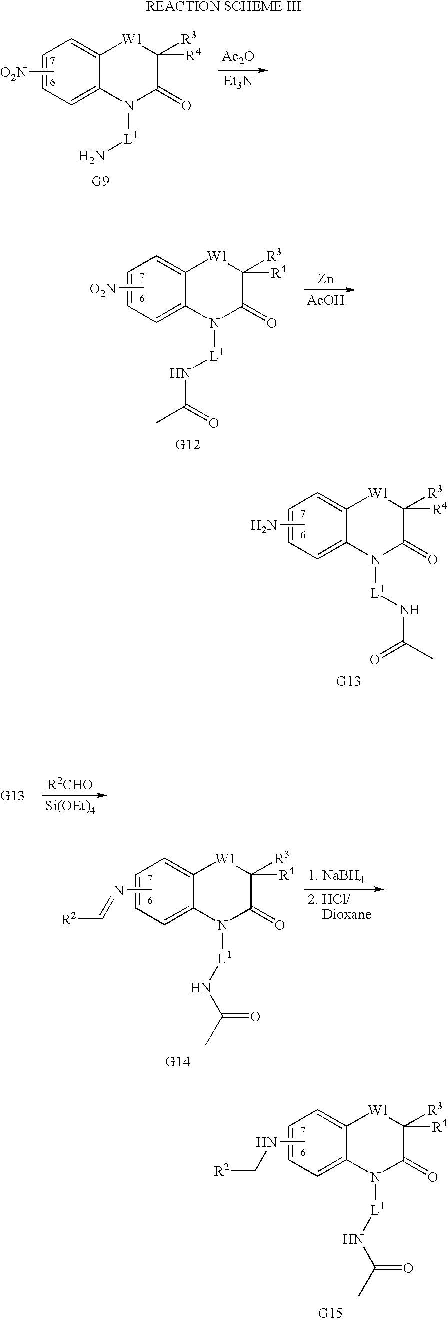

### Step 1—Optional Protection of the R¹ Moiety

In cases where the L¹X¹R¹ moiety has first been attached to the compound core forming a compound of formula 2, it may be necessary to first protect the R¹ moiety from further reaction. As shown in Reaction Scheme III, a amino group that is pendant on the L¹ moiety of the formula 2 compound G9 may undergo acylation under standard conditions including, but not limited to, acylating reagent (for example, Ac₂O or AcCl) and base such as triethylamine, pyridine, diisopropylethylamine, or potassium carbonate in a suitable solvent to produce compounds of Formula G12.

It is noted that the protecting group need not be removed if it is desirable in the final compound of Formula I.

### Step 2—Conversion of the Nitro Group

Once an R¹ moieties are protected, the nitro moiety on the G7 core compound or the protected intermediate compound of formula 2 is converted into an amino group. Typical methods for converting the nitro group into an amino group include, but are not limited to, hydrogenation on metal catalyst, such as palladium, reaction with metals, such as, for example, tin, or iron, or using sodium ditianate in the presence of sodium carbonate. As shown in Reaction Scheme III, one method of carrying out the transformation of compound of Formula G12 into compound of Formula G13 is zinc in acetic acid.

### Step 3—Coupling of the R²L² Moiety

Coupling of the R²L² moiety may then be carried out in a stepwise process with intermediate formation of a Schiff base like G14 followed by reduction with a reducing agent. Generation of Schiff base requires dehydrating conditions like heating with Dean-Stark trap or presence of anhydrous magnesium sulfate or some other hygroscopic reagent. One method of generating Schiff base includes the use of tetraethyl silicate as a reagent.

Alternatively, reductive amination of aldehydes with anilines can be carried out as a “one-pot” procedure. In such a “one-pot” method, reactants are dissolved in an organic solvent such as THF or methanol (preferred) and stirred for a period of time from 1 h to 24 h.

In either the “one-pot” or stepwise process, subsequent use of reducing reagent such as NaBH₄ (preferred), LiAlH₄, NaBH₃CN, or other, is required, to produce the desired product such as secondary amine G15.

### Further Preparation—Secondary Modification of R¹, R², R³, R⁴

It will be appreciated that secondary modification may be made to one or more of the R¹, R², R³, or R⁴ moieties after the compound of Formula I has been made. For example, synthesis of the compound of Formula I may involve the use of a protecting group on a substituent of the R¹ moiety, R² moiety, R³ Moiety, or R⁴ moiety. Once the protecting group is removed, the substituent of the R¹ moiety, R² moiety, R³ moiety, or R⁴ moiety may be further modified to yield further compounds of Formula I.

### Utility Testing and Administration

The present invention relates to compounds, pharmaceutical compositions and methods of using the compounds and pharmaceutical compositions for the treatment and/or prevention of diseases mediated by SCD. The methods and pharmaceutical compositions are particularly suitable for use in the treatment of diseases related to dyslipidemia and disorders of lipid metabolism, especially diseases related to elevated plasma and tissue lipid levels, such as cardiovascular disease, diabetes, obesity, metabolic syndrome, fatty liver diseases and the like.

In general, the compounds of the invention find utility in the treatment of a patient for, or protecting a patient from developing, a disease related to dyslipidemia and/or a disorder of lipid metabolism, wherein lipid levels in an animal, especially a human being, are outside the normal range (i.e., abnormal lipid level, such as elevated plasma or tissue lipid levels), typically where said lipid is a fatty acid, such as a free or complexed fatty acid, triglycerides, phospholipids, wax esters, or cholesterol, such as where VLDL, hepatic or peripheral tissue triglycerides are elevated, or any combination of these, where said lipid-related condition or disease is an SCD-mediated disease or condition such as metabolic syndrome, diabetes, non-alcoholic fatty liver disease, obesity, cancer, oily skin and related diseases, comprising administering to an animal, such as a mammal, especially a human patient, a therapeutically effective a mount of a compound of the invention or a pharmaceutical composition comprising a compound of the invention wherein the compound inhibits the activity of SCD.

The general value of the compounds of the invention in inhibiting the activity of SCD can be determined using the assay described below in Example 6. Additionally, the general value of the compounds in treating disorders and diseases may be established in industry standard animal models for demonstrating the efficacy of compounds in treating obesity, metabolic syndrome, diabetes or abnormal triglyceride or cholesterol levels or for improving glucose tolerance.

### Utility

The compounds of the instant invention are inhibitors of SCD and are useful for treating diseases and disorders in humans and other organisms, including all those human diseases and disorders which are the result of aberrant SCD biological activity or which may be ameliorated by inhibition of SCD biological activity.

As defined herein, an SCD-mediated disease or condition includes but is not limited to a disease or condition which is, or is related to, cardiovascular disease, dyslipidemias, coronary artery disease, atherosclerosis, heart disease, cerebrovascular disease (including, but not limited, to stroke, ischemic stroke and transient ischemic attack (TIA), peripheral vascular disease, and ischemic retinopathy), cancers and oily skin.

Dyslipidemia, as used herein, includes, but is not limited to, disorders related to the serum levels of triglycerides, i.e., hypertriglyceridemia, LDL, VLDL, and/or HDL, cholesterol, and total cholesterol. Dyslipidemia also includes disorders related to the fatty acid Desaturation Index (e.g. the ratio of SCD product fatty acids/SCD substrate fatty acids). Disorders related to polyunsaturated fatty acid (PUFA) are also included as are cholesterol disorders such as familial combined hyperlipidemia and those disorders involving defective reverse cholesterol transport.

SCD-mediated diseases or conditions relating to hypertriglyceridemia include, but are not limited to, hyperlipoproteinemias, familial histiocytic reticulosis, lipoprotein lipase deficiency, apolipoprotein deficiency (such as ApoCII deficiency or ApoE deficiency), and the like, or hypertriglyceridemia of unknown or unspecified etiology.

Metabolic syndrome and Syndrome X are also within the scope of the term “SCD-mediated disease” including all of the various component conditions that make up the syndromes such as, but not limited to, dyslipidemia, low HDL, obesity, insulin resistance, decreased glucose tolerance, hypertension, microalbuminemia, hyperuricaemia, and hypercoagulability, diabetes, non-insulin-dependent diabetes mellitus, Type I diabetes, Type II diabetes, diabetic complications, body weight disorders such as overweight, cachexia and anorexia, and body mass index and leptin related diseases.

As used herein, the term “metabolic syndrome” is a recognized clinical term used to describe a condition comprising combinations of Type II diabetes, impaired glucose tolerance, insulin resistance, hypertension, obesity, increased abdominal girth, hypertriglyceridemia, low HDL, hyperuricaemia, hypercoagulability and/or microalbuminemia.

An SCD-mediated disease or condition also includes various hepatic conditions such as hepatitis, hepatic steatosis, hepatic fibrosis, hepatic cirrhosis, non-alcoholic hepatitis, non-alcoholic steatohepatitis (NASH), alcoholic hepatitis, fatty liver, acute fatty liver, fatty liver of pregnancy, drug-induced hepatitis, erythrohepatic protoporphyria, iron overload disorders, hereditary hemochromatosis, hepatoma and conditions related thereto.

Various skin and mucosal tissue disorders fall within the scope of an SCD-mediated disease or condition including, but not limited to, eczema, acne, psoriasis, keloid scar formation or prevention, diseases related to production or secretions from mucous membranes, such as monounsaturated fatty acids, wax esters, and the like. Inflammation, sinusitis, asthma, pancreatitis, osteoarthritis, rheumatoid arthritis, cystic fibrosis, and pre-menstrual syndrome may also be considered SCD-mediated diseases or conditions as may diseases or conditions which is, or is related to cancer, neoplasia, malignancy, metastases, tumors (benign or malignant), carcinogenesis, hepatomas and the like. SCD-mediated diseases or conditions also include diseases or conditions which are, or are related to, neurological diseases, psychiatric disorders, multiple sclerosis, eye diseases, and immune disorders. An SCD-mediated disease or condition also includes a disease or condition which is, or is related to, viral diseases or infections.

An SCD-mediated disease or condition also includes a condition where increasing lean body mass or lean muscle mass is desired, such as is desirable in enhancing performance through muscle building. Myopathies and lipid myopathies such as carnitine palmitoyltransferase deficiency (CPT I or CPT II) are also included herein. Such treatments are useful in humans and in animal husbandry, including for administration to bovine, porcine or avian domestic animals or any other animal to reduce triglyceride production and/or provide leaner meat products and/or healthier animals.

### Testing

The identification of compounds of the invention as SCD inhibitors was readily accomplished using the SCD enzyme and microsomal assay procedure described in Talamo and Bloch (1969) Analytical Biochemistry 29:300-304. When tested in this assay, compounds of the invention had less than 50% remaining SCD activity at 10 μM concentration of the test compound, typically less than 40% remaining SCD activity at 10 μM concentration of the test compound, for example less than 30% remaining SCD activity at 10 μM concentration of the test compound, such as less than 20% remaining SCD activity at 10 μM concentration of the test compound, thereby demonstrating that the compounds of the invention are potent inhibitors of SCD activity.

Other methods of testing the compounds disclosed herein are also readily available to those skilled in the art. Thus, in addition, testing of the compounds may be accomplished in vivo. In one such embodiment, testing of the compounds is accomplished by administering the compound to an animal afflicted with a plasma or tissue, fatty acid or triglyceride (TG) related disorder or very low density lipoprotein (VLDL)-related disorder and subsequently detecting a change in plasma or tissue fatty acid composition or triglyceride level in said animal thereby identifying a therapeutic agent useful in treating a plasma or tissue, fatty acid or triglyceride (TG) related disorder or very low density lipoprotein (VLDL)-related disorder. In such embodiment, the animal may be a human, such as a human patient afflicted with such a disorder and in need of treatment of said disorder.

In specific embodiments of such in vivo processes, said change in SCD activity in said animal is a decrease in activity, typically wherein said SCD modulating agent does not substantially directly inhibit the biological activity of a Δ5 desaturase, Δ6 desaturase, or fatty acid synthetase or other lipogenic enzymes.

The model systems useful for compound evaluation may include, but not limited to, the use of liver microsomes, such as from mice or rats that have been maintained on a high carbohydrate or high-fat diet, or from human donors, including persons suffering from obesity. Immortalized cell lines, such as HepG2 (from human liver), MCF-7 (from human breast cancer) and 3T3-L1 (from mouse adipocytes) may also be used. Primary cell lines, such as primary hepatocytes and adipocytes, are also useful in testing the compounds of the invention. Where whole animals are used, mice or rats used as a source of primary hepatocyte cells may also be used wherein the mice or rats have been maintained on a high carbohydrate or or other SCD inducing diet to increase SCD activity in microsomes and/or to elevate plasma triglyceride levels or Δ9 fatty acid desaturation indexes (i.e., the 18:1/18:0 ratio); alternatively mice on a normal diet or mice with normal triglyceride levels may be used. Mouse models employing transgenic mice designed for hypertriglyceridemia are also available. Rabbits, hamsters and monkeys are also useful as animal models, especially those with diabetic and obesity phenotypes.

Another suitable method for determining the in vivo efficacy of the compounds of the invention is to indirectly measure their impact on inhibition of SCD enzyme by measuring changes in fatty acid composition. These include absolute or relative reductions in SCD product fatty acids such as 16:1 n-7, 18:1 n-7 or 18:1 n-9. As well fatty acid composition data may also be used to determine a subject's □9 Desaturation Index after administration of the compound. “Desaturation Index(s)” as employed in this specification means the ratio of the product over the substrate for the SCD enzyme as measured from a given tissue sample. This may be calculated using different equations, such as 18:1n-9/18:0; 16:1n-7/16:0; and/or (16:1n-7+18:1n-7)/16:0. Desaturation Index(s) maybe measured in plasma or tissues as well as specific lipid classes containing fatty acids such as triglycerides and phospholipids.

### Administration

The compounds of Formula I may be administered in either single or multiple doses by any of the accepted modes of administration of agents having similar utilities, for example as described in those patents and patent applications incorporated by reference, including buccal, intranasal, intra-arterial injection, intravenously, intraperitoneally, parenterally, intramuscularly, subcutaneously, orally, or as an inhalant.

Oral administration is a typical route for administration of the compounds of Formula I. Administration may be via capsule or enteric coated tablets, or the like. In making the pharmaceutical compositions that include at least one compound of Formula I, the active ingredient is usually diluted by an excipient and/or enclosed within such a carrier that can be in the form of a capsule, sachet, paper or other container. When the excipient serves as a dilutent, in can be a solid, semi-solid, or liquid material (as above), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 20% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, cyclodextrins, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents.

The compositions of the invention can be formulated so as to provide quick, sustained or delayed release of the active ingredient after administration to the patient by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902514; and 5,616,345.

Another formulation for use in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

SCD inhibitors such as the compounds of Formula I are effective over a wide dosage range and are generally administered in a pharmaceutically effective amount. Typically, for oral administration, each dosage unit contains from 1 mg to 2 g of an SCD inhibitor, more commonly from 1 to 700 mg, and for parenteral administration, from 1 to 700 mg of a stearoyl-CoA desaturase inhibitor, more commonly about 2 to 200 mg. It will be understood, however, that the amount of the SCD inhibitor actually administered will be determined by a physician, in the light of the relevant circumstances, including the condition to be treated, the chosen route of administration, the actual compound administered and its relative activity, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules.

The tablets or pills of the present invention may be coated or otherwise compounded to provide a dosage form affording the advantage of prolonged action, or to protect from the acid conditions of the stomach. For example, the tablet or pill can comprise an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Typically the compositions are administered by the oral or nasal respiratory route for local or systemic effect. Compositions in pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, e.g. orally or nasally, from devices that deliver the formulation in an appropriate manner.

The following examples are included to demonstrate typical embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### A. Preparation of Materials, Part 1

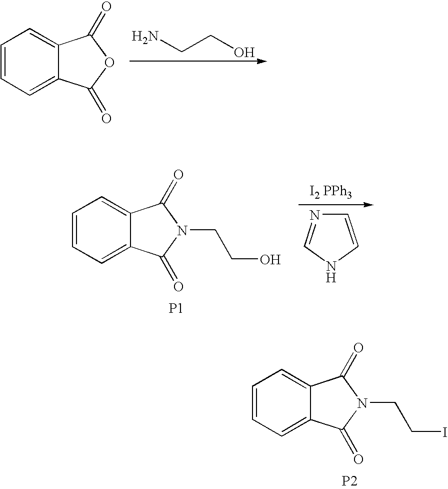

2-(2-Hydroxyethyl)isoindoline-1,3-dione (P1) was synthesized by the published procedure: Organic Syntheses, Coll. Vol. 4, p. 106 (1963).

2-(2-Iodoethyl)isoindoline-1,3-dione (P2). To a cooled to −10° C. suspension of imidazole (16.3 g, 0.24 mol) and PPh₃ (31.5 g, 0.12 mol) in dry CH₂Cl₂ (160 mL) I₂ (30.5 g, 0.12 mol) was added in portions. After the addition the formed suspension was stirred for 30 min at room temperature followed by addition of P1 (15.3 g, 0.08 mol) in portions. The reaction mixture was stirred for 24 h, then aqueous Na₂S₂O₃ was added. Organic layer was consequently washed with water, brine, and dried over Na₂SO₄. After evaporation yellow-green residue was obtained, which was chromatographed (CH₂Cl₂/hexanes 1:2) on silica gel. Recrystallization of the final product from hexanes/acetone yielded 20.9 g (87%) of white fibers.

### B. Preparation of Compounds Analogous to P2

The synthesis scheme of Example 1A may be altered to generate compounds analogous to P2, but varying the iodoalkyl group. For example, in the first reaction of the scheme of Example 1A, the ethanolamine is a C₂ alcohol with an amino substituent and may be replaced with other amino-substituted alkyl alcohols, for example amino-substituted C₃ or C₄ alcohols, such as 3-aminopropan-1-ol, 2-aminopropan-1-ol, 3 aminopropan-2-ol, 4-aminobutan-1-ol, 3-aminobutan-1-ol, 4-aminobutan-2-ol, or 3-aminobutan-2-ol to generate the corresponding analog compounds to P2 with varying iodoalkyl groups. Still other amino-substituted alkyl alcohols may be used to generate the corresponding analog compounds to P2. These analog compounds may then be used to generate corresponding compounds of Formula I by use of the syntheses described herein with the analog compounds.

### EXAMPLE 2

### A. Preparation of Materials, Part 2

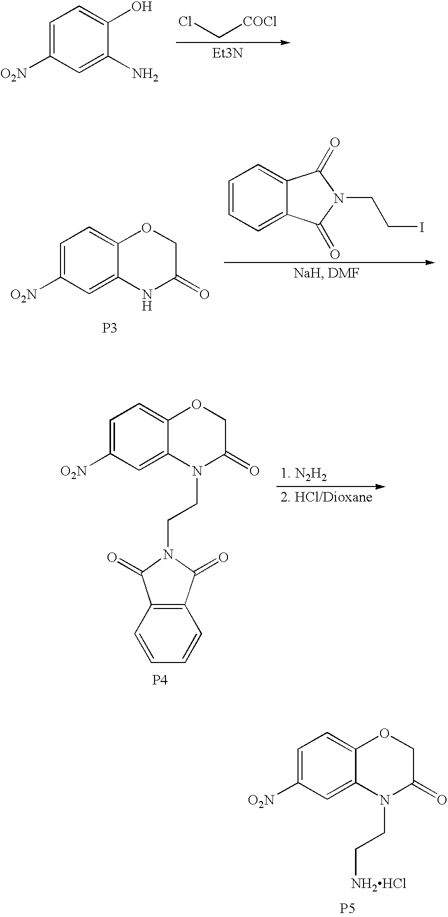

6-Nitro-4H-benzo[1,4]oxazin-3-one (P3). To a cooled to 0° C. solution of 2-amino-4-nitrophenol (4 g, 26 mmol) in acetone (50 mL) chloroacetyl chloride (3.3 g, 29 mmol) was added dropwise. A slight warming-up and precipitation were observed. Then Et₃N (5.8 g, 57 mmol) was slowly added and the reaction mixture was additionally stirred for 30 minutes. Afterwards, the mixture was diluted with water (50 mL) and heated to reflux for 3 hours (h). The product precipitated from the reaction mixture. The mixture was kept overnight at room temperature, filtered, and the residue on filter was consequently washed with excess of water and methanol. After drying, a grey solid (4.3 g, 85%) was obtained.

2-[2-(6-Nitro-3-oxo-2,3-dihydro-benzo[1,4]oxazin-4-yl)-ethyl]-isoindole-1,3-dione (P4). To a cooled to −5° C. solution of P3 (7.4 g, 38 mmol) in dry DMF (90 mL) NaH (3 g, 75 mmol, 60% dispersion in mineral oil) was added slowly under argon atmosphere. After the addition the formed suspension was stirred at room temperature for 1 h, then it was cooled again to −5° C., and P2 (20.5 g, 68 mmol) was added in portions at this temperature. The reaction mixture was additionally stirred at room temperature for 17 hours then at 40° C. for 4 days. Afterwards, the mixture was diluted with ethanol (450 mL) and water (100 mL) and kept at 5° C. overnight. The precipitated product was filtered and washed with ethanol. After drying yellow powder (7.7 g, 55%) was obtained.

4-(2-Aminoethyl)-6-nitro-4H-benzo[1,4]oxazin-3-one (P5). To P4 (7.7 g, 21 mmol) in ethanol (150 mL) N₂H₄*H₂O (10 mL) was added, and the mixture was stirred at 65° C. for 3 hours. The precipitate was filtered off and washed with ethanol. Filtrate was evaporated, the brown residue was dissolved in 5% aq NaOH (100 mL) and CH₂Cl₂ (150 mL). Aqueous layer was additionally extracted with CH₂Cl₂ (4 times), then the organic layers were combined, washed with water, and dried over Na₂SO₄. Evaporation of the solvent yielded 4.4 g of yellow powder. It was dissolved in THF (120 mL) and 4 N HCl in dioxane (8 mL, 32 mmol) was added. The formed precipitate was filtered and washed with THF to give 4.7 g (82%) of beige powder after drying. The obtained product was relatively pure, giving one major spot by TLC (CH₂Cl₂/EtOH 5%) with R_{f}=0.45. It was used in further steps without additional purification.

### B. Preparation of Compounds Analogous to P5

The synthesis scheme of Example 2A may be altered to generate compounds analogous to P3. As an example, in the first reaction of the scheme of Example 2, the 2-amino-4-nitrophenol may be replaced with 2-amino-5-nitrophenol and/or the chloroacetyl chloride may be replaced with 2-bromo-2-methylpropanoyl bromide to generate the corresponding analog compounds to P3. These analog compounds to P3 may then be used to generate corresponding compounds of Formula I by use of the syntheses described herein with the analog compounds.

### EXAMPLE 3

A. Preparation of a Compound of Formula I in which R² is Optionally Substituted phenyl and L² is methylene.

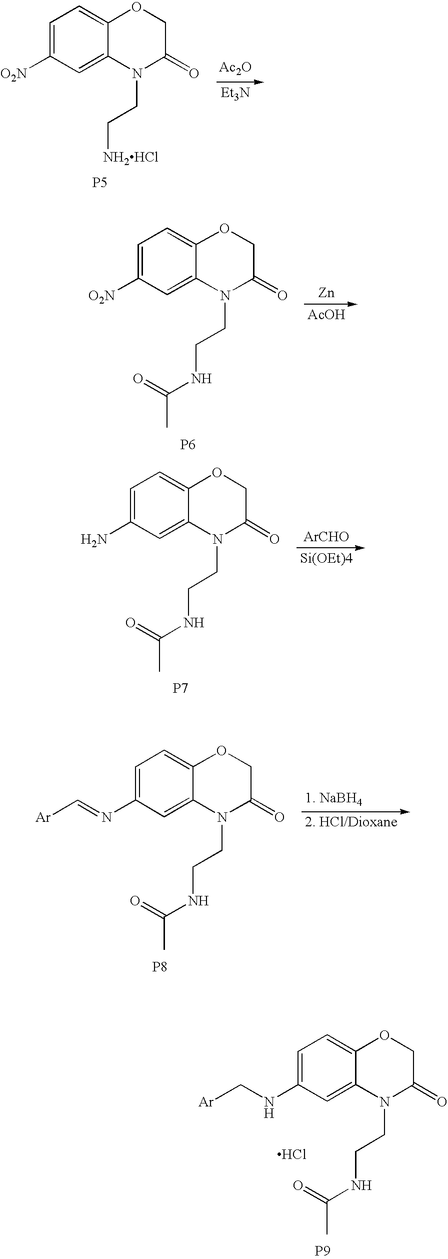

N-[2-(6-Nitro-3-oxo-2,3-dihydro-benzo[1,4]oxazin-4-yl)-ethyl]-acetamide (P6). To a cooled to −5° C. solution of P5 (2 g, 7.3 mmol) and triethylamine (Et₃N) (5.8 g, 57 mmol) in CH₂Cl₂ (50 mL) acetic anhydride (AC₂O) (2.2 g, 22 mmol) was added in one portion. The reaction mixture was stirred at room temperature for 3 hours, then 5% aq HCl was added. The formed precipitate was filtered off and rinsed with water to give 0.7 g of beige solid after drying. Organic layer from the filtrate was consequently washed with water, aqueous Na₂CO₃, brine, and dried over Na₂SO₄. Evaporation of CH₂Cl₂ gave additional yellow solid.

N-[2-(6-Amino-3-oxo-2,3-dihydro-benzo[1,4]oxazin-4-yl)-ethyl]-acetamide (P7). A suspension of P6 (200 mg, 0.7 mmol) and Zn (650 mg, 10 mmol) in acetic acid (AcOH) (10 mL) was stilted at 50-55° C. for 3.5 h. AcOH was then evaporated, and the residue was taken into boiling CH₂Cl₂ and filtered. This operation was repeated with the precipitate 4 times. The combined organic phase was consequently washed with aqueous Na₂CO₃, water, brine, and dried over Na₂SO₄. Evaporation of CH₂Cl₂ gave tan solid. The obtained product was relatively pure, giving one major spot by TLC (Ethyl acetate/EtOH 5%) with R_{f}=0.35. It was used in further steps without additional purification.

General Procedure for the Schiff base formation (P8). A solution of P7 (0.27 g, 1.1 mmol), Si(OEt)₄ (1.1 g, 5.3 mmol), and the corresponding benzaldehyde (1.3 mmol) in absolute ethanol (5 mL) was refluxed for 4.5 h. The Schiff base crystallized from the solution on slow cooling to 4° C. It was filtered and washed with cold ethanol on filter. Additional product could be obtained by partial evaporation of the filtrate and subsequent cooling. TLC analysis showed high purity of the desired product. It was used in further steps without additional purification. This general procedure for Schiff base formation was employed to generate the following products:

N-[2-[6-(Benzylideneamino)-3-oxo-2,3-dihydro-benzo[1,4]oxazin-4-yl]-ethyl]acetamide. R_{f}=0.45 (CH₂Cl₂/EtOH 5%).

N-(2-[6-[(3,4-Dichlorobenzylidene)amino]-3-oxo-2,3-dihydro-benzo[1,4]oxazin-4-yl]-ethyl)acetamide. R_{f}=0.75 (Ethyl acetate/EtOH 5%).

N-(2-[6-[[4-Chloro-3-(trifluoromethyl)benzylidene]amino]-3-oxo-2,3-dihydro-benzo[1,4]oxazin-4-yl]-ethyl)acetamide. R_{f}=0.35 (CH₂Cl₂/EtOH 5%).

N-(2-[6-[(4-Fluoro-3-(trifluoromethyl)benzylidene]amino]-3-oxo-2,3-dihydro-benzo[1,4]oxazin-4-yl]-ethyl)acetamide. R_{f}=0.3 (CH₂Cl₂/EtOH 5%).

(E)-N-(2-(7-(3,4-dichlorobenzylideneamino)-3-oxo-2H-benzo[b][1,4]oxazin-4(3H)-yl)ethyl)acetamide.

(Z)-N-(2-(6-(3,4-dichlorobenzylideneamino)-2,2-dimethyl-3-oxo-2H-benzo[b][1,4]oxazin-4(3H)-yl)ethyl)acetamide.

General Procedure for the Schiff Base Reduction, generating products of Formula I (P9). To a cooled to −5° C. suspension of P8 (0.7 mmol) in absolute ethanol (5 mL) NaBH₄ (76 mg, 2 mmol) was added in one portion followed by dry THF until the solution became homogeneous. It was stirred at room temperature until TLC analysis indicated no starting Schiff base left in the reaction. Then the solvents were evaporated, and the residue was dissolved in water and ethyl acetate. Organic layer was consequently washed with water and brine, dried over Na₂SO₄, and evaporated leaving a crude product. It was further purified by flash chromatography on silica gel eluting with CH₂Cl₂, gradually changing the eluent to ethyl acetate. All fractions containing the pure product by TLC were combined and evaporated leaving yellow solid, which was recrystallized from CH₂Cl₂/Hexanes. The crystalline product was dissolved in THF (10 mL), and 4 N HCl in dioxane (0.25 mL, 1 mmol) was added with stirring. The formed precipitate was filtered, washed on filter with THF or ether, and dried to give a target product as beige powder. The product of the reaction, P9, is a compound of Formula I in which R¹ is 3,4-dichlorophenyl and L² is methylene.

This general procedure for Schiff base reduction was employed to generate the following products of Formula I:

N-(2-(6-(benzylamino)-3-oxo-2H-benzo[b][1,4]oxazin-4(3H)-yl)ethyl)acetamide hydrochloride.

N-(2-(6-(3,4-dichlorobenzylamino)-3-oxo-2H-benzo[b][1,4]oxazin-4(3H)-yl)ethyl)acetamide hydrochloride.

N-(2-(6-(4-chloro-3-(trifluoromethyl)benzylamino)-3-oxo-2H-benzo[b][1,4]oxazin-4(3H)-yl)ethyl)acetamide hydrochloride.

N-(2-(6-(4-fluoro-3-(trifluoromethyl)benzylamino)-3-oxo-2H-benzo[b][1,4]oxazin-4(3H)-yl)ethyl)acetamide hydrochloride.

N-(2-(7-(3,4-dichlorobenzylamino)-3-oxo-2H-benzo[b][1,4]oxazin-4(3H)-yl)ethyl)acetamide.

N-(2-(6-(3,4-dichlorobenzylamino)-2,2-dimethyl-3-oxo-2H-benzo[b][1,4]oxazin-4(3H)-yl)ethyl)acetamide.

### B. Preparation of Compounds of Formula (I) Varying R²

Similarly, following the procedure of Example 3A above, but optionally substituting other compounds having the structure ArCHO (varying the Ar group, where Ar is an optionally substituted aryl group), other compounds of Formula I are prepared.

### EXAMPLE 4

A. Preparation of a Compound of Formula I in which RE is Optionally Substituted phenyl, R² is Optionally Substituted phenyl and L² is methylene.

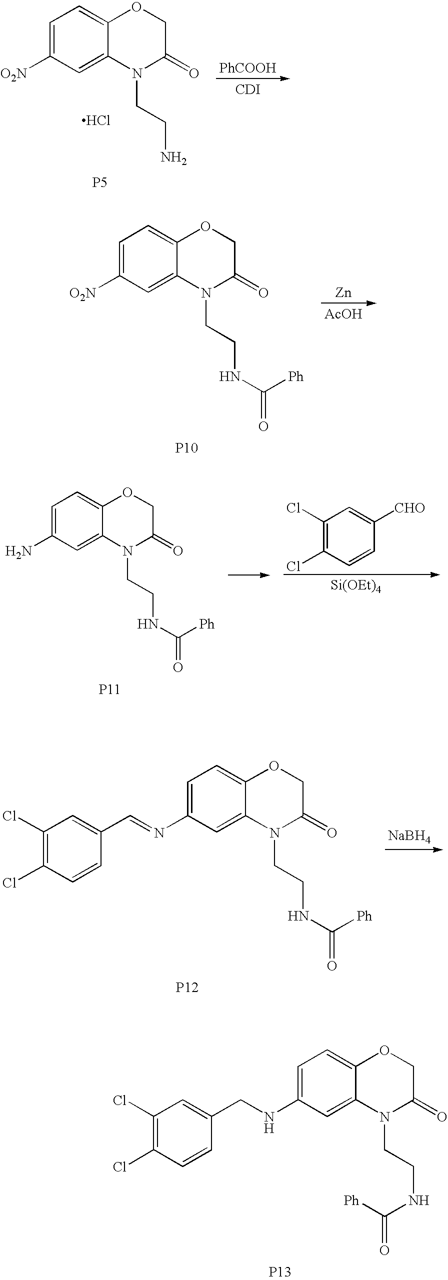

N-[2-(6-Nitro-3-oxo-2,3-dihydro-benzo[1,4]oxazin-4-yl)-ethyl]-benzamide (P10). A solution of benzoic acid (0.3 g, 2.5 mmol) and carbonyldiimidazole (CDI) (0.4 g, 2.5 mmol) in dry THF (10 mL) was stirred at room temperature for 2 h. PS (0.55 g, 2 mmol) was added in one portion, and the suspension was stirred at room temperature for 12 h then refluxed for 9 h. Additionally a solution of premixed benzoic acid (0.15 g, 1.2 mmol) and CDI (0.19 g, 1.2 mmol) in dry THF (4 mL) was added. After reflux for 9 h some of the starting amine still could be detected by TLC analysis. A solution of premixed benzoic acid (0.3 g, 2.5 mmol) and CDI (0.4 g, 2.5 mmol) in absolute THF (7 mL) was added again, followed by Et₃N (0.2 g, 2 mmol). The dark reaction mixture was stirred at room temperature overnight then refluxed for 5 hours. At this point no starting material was observed by TLC. THF was evaporated, and the residue was stirred in aqueous HCl, filtered, washed on filter consequently with water, aqueous Na₂CO₃, water. After drying on air the precipitate was extracted with boiling acetone. Evaporation of the filtrate gave light-brown solid, which was recrystallized from ethanol to give a beige powder.

N-[2-(6-Amino-3-oxo-2,3-dihydro-benzo[1,4]oxazin-4-yl)-ethyl]-benzamide (P11). The suspension of P10 (0.34 g, 1 mmol) and Zn (0.85 g, 13 mmol) in AcOH (13 mL) was stirred at 50-55° C. for 3.5 h. AcOH was then evaporated, and the residue was taken into boiling CH₂Cl₂ and filtered. This operation was repeated with the precipitate 4 times. Combined organic layer was consequently washed with aqueous Na₂CO₃, water, brine, and dried over Na₂SO₄. Evaporation of CH₂Cl₂ gave 0.31 g (99%) of tan solid. The obtained product was relatively pure, giving one major spot by TLC (CH₂Cl₂/EtOH 5%) with R_{f}=0.25. It was used in further steps without additional purification.

N-[2-[6-[(3,4-Dichlorobenzylidene)amino]-3-oxo-2,3-dihydro-benzo[1,4]oxazin-4-yl]-ethyl]benzamide (P12). A solution of P11 (308 mg, 1 mmol), Si(OEt)₄ (1.1 g, 5.3 mmol), and 3,4-dichlorobenzaldehyde (210 mg, 1.2 mmol) in absolute ethanol (5 mL) was refluxed for 5 h. The reaction mixture was cooled to 4° C., and the precipitate was filtered and washed with cold ethanol on filter to give 313 mg (67%) of beige powder after drying. The obtained product was relatively pure, giving one major spot by TLC (CH₂Cl₂/EtOH 5%) with R_{f}=0.35. It was employed without additional purification.

N-(2-(6-(3,4-dichlorobenzylamino)-3-oxo-2H-benzo[b][1,4]oxazin-4(3H)-yl)ethyl)benzamide (P13). To a cooled to −5° C. suspension of P12 (310 mg, 0.67 mmol) in absolute ethanol (6 mL) NaBH₄ (75 mg, 2 mmol) was added in one portion followed by dry THF until the solution became homogeneous. It was stirred at room temperature until TLC analysis indicated no starting Schiff base left in the reaction. At this point, the solvents were evaporated, and the residue was dissolved in water and CH₂Cl₂. Organic layer was consequently washed with water and brine, dried over Na₂SO₄, and evaporated leaving light-brown oil. It was subjected to flash chromatography on silica gel eluting with CH₂Cl₂, gradually changing the eluent to ethyl acetate. All fractions containing the pure product by TLC were combined and evaporated leaving grey solid, which was recrystallized two times from CH₂Cl₂/Et₂O/hexanes and CH₂Cl₂/hexanes. After drying in vacuo beige powder was obtained.

### B. Preparation of Compounds of Formula (I) Varying R¹ and R²

Similarly, following the procedure of Example 4A above, but optionally replacing some of the materials used, other compounds of Formula I are prepared. For example, in the first reaction of the scheme of Example 4A, the benzoic acid may be replaced with an optionally substituted benzoic acid or other aryl group having a carboxyl substituent to generate the corresponding compounds of Formula I. As another example, in the third reaction of the scheme of Example 2, the dichlorobenzaldehye may be replaced with other optionally substituted benzaldehyde compounds to generate the corresponding compounds of Formula I.

### EXAMPLE 5

A. Preparation of a Compound of Formula I in which R¹ is 3,4-dichlorophenyl and R² is hydroxymethyl.

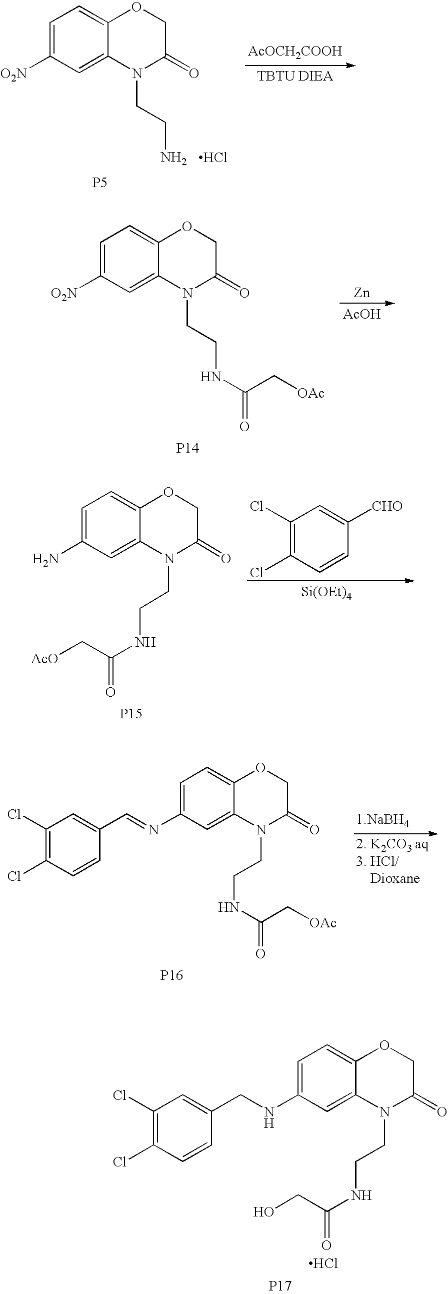

Acetic acid [2-(6-nitro-3-oxo-2,3-dihydro-benzo[1,4]oxazin-4-yl)-ethylcarbamoyl]methyl ester (P14). A suspension of acetoacetic acid (0.22 g, 1.9 mmol), diisopropylethyl amine (DIEA) (0.26 g, 2 mmol), and O-(Benzotriazol-1-yl)-N,N,N′N′-tetramethyluronium tetrafluoroborate (TBTU) (0.61 g, 1.9 mmol) in absolute THF (10 mL) was stirred at room temperature overnight. Additional diisopropylethyl amine (0.26 g, 2 mmol) was added to a cooled to 0° C. reaction mixture followed by P5 (0.46 g, 1.7 mmol). After stirring for 4 h at room temperature no starting material was detected by TLC. THF was evaporated, the residue was stirred in dilute aqueous HCl, filtered, and subsequently washed on filter with water, aqueous NaHCO₃, water. After drying yellow powder was obtained. The obtained product was relatively pure, giving one major spot by TLC (CH₂Cl₂/EtOH 5%) with R_{f}=0.5. It was used in further steps without additional purification.

Acetic acid [2-(6-amino-3-oxo-2,3-dihydro-benzo[1,4]oxazin-4-yl)-ethylcarbamoyl]methyl ester (P15). A suspension of P14 (0.4 g, 1.2 mmol) and Zn (1.05 g, 16 mmol) in AcOH (15 mL) was stirred at 50-55° C. for 3.5 h. AcOH was then evaporated, and the residue was taken into boiling CH₂Cl₂ and filtered. This operation was repeated with the precipitate 4 times. Combined organic layer was subsequently washed with aqueous NaHCO₃, water, brine, and dried over Na₂SO₄. Evaporation of CH₂Cl₂ gave a grey solid. The obtained product was relatively pure, giving one major spot by TLC (CH₂Cl₂/EtOH 5%) with R_{f}=0.3. It was used in further steps without additional purification.

N-[2-[6-(3,4-Dichlorobenzylamino)-3-oxo-2,3-dihydro-benzo[1,4]oxazin-4-yl]-ethyl]-2-hydroxyacetamide hydrochloride (P17). A solution of P15 (364 mg, 1.2 mmol), Si(OEt)₄ (1.6 g, 7.7 mmol), and 3,4-dichlorobenzaldehyde (245 mg, 1.4 mmol) in absolute ethanol (7 mL) was refluxed for 3 h. At this point, TLC analysis showed predominately the Schiff base P16 with R_{f}=0.6 (ethyl acetate), no starting amine P15 was observed. The reaction mixture was cooled to 0° C., and NaBH₄ (46 mg, 1.2 mmol) was added in one portion followed by dry THF until the solution became homogeneous. It was stirred at room temperature until TLC analysis indicated no starting Schiff base left in the reaction. Solvents were evaporated, and the residue was dissolved in aqueous methanol. K₂CO₃ (124 mg, 0.9 mmol) was added, and the suspension stirred for 1 h. The reaction mixture was concentrated in vacuo and filtered through a silica gel plug eluting with CH₂Cl₂, changing the eluent to ethyl acetate. All fractions containing the product by TLC were combined and evaporated leaving yellow oily solid. It was dissolved in THF/Et₂O, and 4 N HCl in dioxane (0.3 mL, 1.2 mmol) was added with stirring. The formed precipitate was filtered and washed on filter with THF. After drying it was dissolved on stirring in ethyl acetate and aqueous Na₂CO₃. Organic layer and the extractions of the aqueous phase were combined, dried over Na₂SO₄, and evaporated. The residue was subjected to flash chromatography on silica gel eluting with CH₂Cl₂, gradually changing the eluent to ethyl acetate. All fractions containing the pure product by TLC were combined and evaporated leaving light-yellow solid. It was refluxed in Et₂O for 1 h, afterwards the suspension was cooled to 4° C., and the precipitate was filtered and washed with cold Et₂O. It was dissolved in THF, and to the formed solution 4 N HCl in dioxane (0.15 mL, 0.6 mmol) was added with vigorous stirring. The precipitate was filtered, washed with cold THF and dried to give beige powder.

This general procedure was employed to generate the following products of Formula I:

N-(2-(7-(3,4-dichlorobenzylamino)-3-oxo-2H-benzo[b][1,4]oxazin-4(3H)-yl)ethyl)-2-hydroxyacetamide

N-(2-(6-(3,4-dichlorobenzylamino)-2,2-dimethyl-3-oxo-2H-benzo[b][1,4]oxazin-4(3H)-yl)ethyl)-2-hydroxyacetamide

### B. Preparation of Compounds of Formula I Varying R¹ and R²

Similarly, following the procedure of Example 5SA above, but optionally substituting other compounds in place of the acetoacetic acid and/or in place of the dichlorobenzaldehyde (e.g. other substituted benzaldehydes, other compounds of formula Ar—C(O)H where Ar is optionally substituted aryl), other compounds of Formula I are prepared.

### EXAMPLE 6

### Characterization of Stearoyl-CoA Desaturase Inhibitor

### Materials and Methods

### Materials

[³H]stearoyl CoA and sterculic acid were obtained from PerkinElmer and Planta Piloto de Quimica Fina, respectively. Commercial sources of other reagents are listed below:

| Material | Company |
|---|---|
| [³H]H₂O | PerkinElmer |
| Stearoyl CoA | Sigma |
| CoA | Sigma |
| NADH | Sigma |
| Tris, 1M | Invitrogen |
| MgCl2 | Sigma |
| BHT | Sigma |
| BSA | Sigma |
| DMSO | Sigma |
| ATP | Sigma |
| 96-well plates | Corning |
| Bio-Beads SM-2 | Bio-Rad |

### Preparation of Rat Liver Microsomes

The rat liver microsomes were collected according to the procedure described in Ozols (1990) Methods Enzm, 182:225.

In vivo Experiment (Liver Perfusion and Collection)

Male Sprague Dawley Rats were placed on regimented fasting protocol for one week to stimulate SCD enzymatic activity. 48-hour periods were alternated between feeding and fasting to induce and down-regulate SCD activity with SCD activity being induced via carbohydrate rich diet prior to liver perfusion and collection.

The rats were anesthetized with Isoflurane inhalation anesthetic, the liver perfused with cold phosphate buffered saline (PBS), weighed, and chilled in cold homogenization buffer (250 mM sucrose, 10 mM Tris, 1 mM EDTA, pH 7.6).

The livers were finely minced and placed in homogenization tube. Homogenization buffer (40 mL) was added to the homogenization tube and the liver homogenized and centrifuged in a pre-chilled SLA-600 TC at 800G rotor for 10 min at 4° C.

Following centrifugation, the supernatant was collected and the pellet removed and discarded. The supernatant was centrifuge at 10,000 G for 35 minutes. Following centrifugation, the supernatant was collect and the pellet discarded. The supernatant was then centrifuged in a pre-chilled 45-Ti rotor at 130,000 G (41,000 RPM) for 90 minutes at 4° C.

In vitro (Microsomal Collection)

The supernatant was then aspirated off and the collected microsomal pellet washed in 25 mL of Glycerol PBS (1× PBS 7.4, 20% Glycerol) and resuspended in 4-5 volumes of Glycerol PBS.

The protein concentration of the microsomal preparation was determined by BCA assay (Pierce) and the microsomes were aliquoted and stored at −80° C.

### Preparation of Hydrophobic Beads

Biobeads were ground to a smaller size in a mortar and pestle and resuspended in 3.6% TCA. The beads were then filtered through 300 μM mesh.

### Stock Solutions

Stock solutions and their storage conditions are listed below:

| Solution | Storage condition |
|---|---|
| 20 mg/ml Stearoyl CoA | −80° C. |
| 2.8 mCi/ml [³H]Stearoyl CoA | −80° C. |
| CoA | freshly prepared |
| Sterculic acid | freshly prepared |
| 0.2 M NADH | −80° C. |
| 1 M Tris, pH 7.2 | room temperature |
| 1 M MgCl2 | room temperature |
| 100 mM ATP | −20° C. |
| 10% BSA | 4° C. |
| 10-20 mg/ml microsome | −80° C. |

### The SCD Assay Buffer

SCD was determined in the desaturase assay buffer. This assay buffer contained 0.1 M Tris buffer, pH 7.2, 2 mM NADH, 4.8 mM ATP, 0.5 mM CoA, 4.8 mM MgCl₂, and 0.1% BSA.

The Procedure for the SCD Assay (Adapted from Talamo and Bloch (1969) Analytical Biochemistry 29:300-304)

1 μl of each compound of Formula I was added to an assay plate by a low volume (0.5-10 μL) multichannel pipette. A DMSO control was also prepared. The microsomes were quickly thawed and added to assay buffer so that a concentration of 0.4 mg/ml was achieved (0.2 mg/ml assay final). 50 μl of the microsome suspension in assay buffer was then added into each well in the compound assay plate, the plate was covered, and the microsomes preincubated with the compounds for 30 minutes on the orbital shaker, 50-75 rpm, at room temperature.

After preincubation, the reaction was initiated by the addition of 50 μl of substrate solution (20 μM Stearoyl CoA, [3H]Stearoyl CoA, 74nCi) to the preincubated microsomes/compound suspensions in MilliQ (Millipore) H₂O. The reaction mixtures were then incubated for 45 minutes on the orbital shaker at 50-75 rpm at room temperature.

The reaction was terminated by the addition of 10 μl of 21% trichloroacetic acid (TCA) to the reaction mixture followed incubation on the orbital shaker for 30 minutes at 50-75 rpm at room temperature followed by centrifugation for 5 minutes at 3700 rpm.

50 μl of a 6% Bio-Bead suspension in H₂O was added to the reaction mixture and the assay plate was sealed. The Bio-Bead mixture was incubated on the orbital shaker for 1 hour, 100-150 rpm at room temperature, and then the mixture was centrifuged at 2000 g for 5 minutes to pellet the Bio-Beads.

25 μl of the supernatant was harvested from each well and transferred to a detection plate. 100 μl of OptiPhase SuperMix scintillation cocktail (containing sufficient NaOH to neutralize the TCA) was added and the solutions mixed by vigorous shaking (300-400 rpm) for 5 minutes at room temperature. The radioactivity was counted in a MicroBeta scintillation counter in order to determine the activity and IC₅₀ values for the compounds of Formula I. Table 1 presents the IC₅₀ data for a number of compounds of the invention for which the IC₅₀ as determined in the above assay was less than 30 μm.

TABLE 1

| | | IC₅₀ |
|---|---|---|
| NUMBER | NAME | μM |
| 1. | 3-(6-(3,4-dichlorobenzylamino)-3-oxo-2H-benzo[b][1,4]oxazin- | 0.0535 |
| | 4(3H)-yl)propanamide | |
| 2. | N-(2-(6-(benzylamino)-3-oxo-2H-benzo[b][1,4]oxazin-4(3H)- | 14.9 |
| | yl)ethyl)acetamide hydrochloride | |
| 3. | N-(2-(6-(3,4-dichlorobenzylamino)-3-oxo-2H-benzo[b][1,4]oxazin- | 0.0107 |
| | 4(3H)-yl)ethyl)acetamide hydrochloride | |
| 4. | 6-(3,4-dichlorobenzylamino)-4-(2-phenoxyethyl)-2H- | 22.2 |
| | benzo[b][1,4]oxazin-3(4H)-one | |
| 5. | N-(2-(6-(4-chloro-3-(trifluoromethyl)benzylamino)-3-oxo-2H- | 0.00487 |
| | benzo[b][1,4]oxazin-4(3H)-yl)ethyl)acetamide hydrochloride | |
| 6. | N-(2-(6-(4-fluoro-3-(trifluoromethyl)benzylamino)-3-oxo-2H- | 0.0078 |
| | benzo[b][1,4]oxazin-4(3H)-yl)ethyl)acetamide hydrochloride | |
| 7. | N-(2-(6-(3,4-dichlorobenzylamino)-3-oxo-2H-benzo[b][1,4]oxazin- | 0.00252 |
| | 4(3H)-yl)ethyl)benzamide | |
| 8. | N-(2-(6-(3,4-dichlorobenzylamino)-3-oxo-2H-benzo[b][1,4]oxazin- | 0.00139 |
| | 4(3H)-yl)ethyl)-2-hydroxyacetamide hydrochloride | |
| 9. | (±)-N-(2-(6-(3,4-dichlorobenzylamino)-2-methyl-3-oxo-2H- | 0.001-0.01 |
| | benzo[b][1,4]oxazin-4(3H)-yl)ethyl)acetamide | |
| 10. | (±)-N-(2-(6-(4-chloro-3-(trifluoromethyl)benzylamino)-2- | 0.001-0.01 |
| | methyl-3-oxo-2H-benzo[b][1,4]oxazin-4(3H)- | |
| | yl)ethyl)acetamide | |
| 11. | (±)-N-(2-(6-(3,4-dichlorobenzylamino)-2-methyl-3-oxo-2H- | 0.08 |
| | benzo[b][1,4]oxazin-4(3H)-yl)ethyl)-2-hydroxyacetamide | |
| 12. | (±)-N-(2-(6-(4-chloro-3-(trifluoromethyl)benzylamino)-2- | 0.001-0.01 |
| | methyl-3-oxo-2H-benzo[b][1,4]oxazin-4(3H)-yl)ethyl)-2- | |
| | hydroxyacetamide | |

We claim:

1. A compound that is an inhibitor of stearoyl-CoA desaturase having the structure of Formula I: 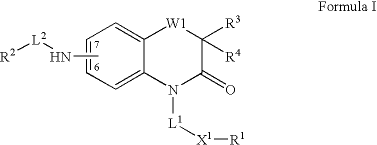
wherein
R¹ is hydrogen, optionally substituted C₁₋₂₀ alkyl, optionally substituted C₁₋₆ lower alkyl, optionally substituted C₃₋₂₀ cycloalkyl, optionally substituted C₂₋₂₀ alkenyl, optionally substituted C₂₋₂₀ alkynyl, optionally substituted C₁₋₂₀ alkoxy, optionally substituted C₁₋₆ alkoxy, optionally substituted mono- or bicyclic heterocyclyl, optionally substituted mono- or bicyclic aryl, or optionally substituted mono- or bicyclic heteroaryl;
R² is C₁₋₂₀ alkyl, optionally substituted mono- or bicyclic heterocyclyl, optionally substituted mono- or bicyclic aryl, or optionally substituted mono- or bicyclic heteroaryl;
R³ and R⁴ are independently hydrogen, optionally substituted C₁₋₆ alkyl, optionally substituted C₂₋₆ alkenyl, optionally substituted C₂₋₆ alkynyl, optionally substituted cycloalkyl, optionally substituted mono- or bicyclic heterocyclyl, optionally substituted mono- or bicyclic aryl, optionally substituted mono- or bicyclic heteroaryl,
X¹ is selected from: —O—C(O)—, —C(O)—O—, —NR′—C(O)—, —C(O)—NR′—, —O—C(O)—NR′—, —NR′—, —O—, —S—, —NR′—S(O)₂—, or —S(O)₂—NR′—, wherein R′ is hydrogen or C₁₋₆ lower alkyl;
L¹ is a covalent bond or -Lk-Y—, wherein Lk is optionally substituted linear or branched C₁₋₄ alkylene and Y is selected from a covalent bond, —O—, —S—, or —NR″—, wherein R″ is hydrogen or C₁₋₆ lower alkyl;
L² is a covalent bond or -Lk′-Y′—, wherein Lk′ is optionally substituted linear or branched C₁₋₄ alkylene and Y′ is selected from a covalent bond, —O—, —S—, or —NR″—, wherein R″ is hydrogen or C₁₋₆ lower alkyl;
W1 is —O— or —S—; and
the R²-L²-NH— is bonded to the 6 or 7 position indicated in Formula I.

2. The compound of claim 1 wherein
R² is phenyl which may be optionally substituted at the 2, 3, 4, or 5 position with 1, 2, or 3 substituents selected from the group consisting of optionally substituted lower alkyl, optionally substituted lower alkoxyl, halogen, CF₃, —OCF₃, —OCF₂CF₃ and —OCH₃; and
L¹ is C₂₋₃ alkylene.

3. The compound of claim 2 wherein X¹ is —NR′—C(O)—.

4. The compound of claim 3, wherein R¹ is optionally substituted lower alkyl or optionally substituted phenyl.

5. The compound of claim 4, selected from the group consisting of:
N-(2-(7-(3,4-dichlorobenzylamino)-3-oxo-2H-benzo[b][1,4]oxazin-4(3H)-yl)ethyl)-2-hydroxyacetamide;
N-(2-(6-(3,4-dichlorobenzylamino)-2,2-dimethyl-3-oxo-2H-benzo[b][1,4]oxazin-4(3H)-yl)ethyl)-2-hydroxyacetamide;
N-(2-(7-(3,4-dichlorobenzylamino)-3-oxo-2H-benzo[b][1,4]oxazin-4(3H)-yl)ethyl)acetamide;
N-(2-(6-(3,4-dichlorobenzylamino)-2,2-dimethyl-3-oxo-2H-benzo[b][1,4]oxazin-4(3H)-yl)ethyl)acetamide;
N-(2-(6-(benzylamino)-3-oxo-2H-benzo[b][1,4]oxazin-4(3H)-yl)ethyl)acetamide;
N-(2-(6-(3,4-dichlorobenzylamino)-3-oxo-2H-benzo[b][1,4]oxazin-4(3H)-yl)ethyl)acetamide;
N-(2-(6-(4-chloro-3-(trifluoromethyl)benzylamino)-3-oxo-2H-benzo[b][1,4]oxazin-4(3H)-yl)ethyl)acetamide;
N-(2-(6-(4-fluoro-3-(trifluoromethyl)benzylamino)-3-oxo-2H-benzo[b][1,4]oxazin-4(3H)-yl)ethyl)acetamide hydrochloride;
N-(2-(6-(3,4-dichlorobenzylamino)-3-oxo-2H-benzo[b][1,4]oxazin-4(3H)-yl)ethyl)benzamide; and
N-(2-(6-(3,4-dichlorobenzylamino)-3-oxo-2H-benzo[b][1,4]oxazin-4(3H)-yl)ethyl)-2-hydroxyacetamide hydrochloride.

6. The compound of claim 2, wherein
R² is optionally substituted phenyl;
L² is methylene, and
L¹ is C₂₋₃ alkylene.

7. The compound of claim 6, selected from the group consisting of:
3-(6-(3,4-dichlorobenzylamino)-3-oxo-2H-benzo[b][1,4]oxazin-4(3H)-yl)propanamide; and
6-(3,4-dichlorobenzylamino)-4-(2-phenoxyethyl)-2H-benzo[b][1,4]oxazin-3(4H)-one.

8. The compound of claim 1, wherein R¹ is optionally substituted lower alkyl or optionally substituted lower alkoxy.

9. The compound of claim 8 wherein
R³ is hydrogen;
R⁴ is optionally substituted C₁₋₆ alkyl; and
X¹ is —NR′—C(O)—, —C(O)—NR′—, or —O—.

10. The compound of claim 9, selected from the group consisting of:
N-(2-(6-(3,4-dichlorobenzylamino)-2-methyl-3-oxo-2H-benzo[b][1,4]oxazin-4(3H)-yl)ethyl)acetamide.
N-(2-(6-(4-chloro-3-(trifluoromethyl)benzylamino)-2-methyl-3-oxo-2H-benzo[b][1,4]oxazin-4(3H)-yl)ethyl)acetamide.
N-(2-(6-(3,4-dichlorobenzylamino)-2-methyl-3-oxo-2H-benzo[b][1,4]oxazin-4(3H)-yl)ethyl)-2-hydroxyacetamide.
N-(2-(6-(4-chloro-3-(trifluoromethyl)benzylamino)-2-methyl-3-oxo-2H-benzo[b][1,4]oxazin-4(3H)-yl)ethyl)-2-hydroxyacetamide.

11. The compound of claim 6, wherein X¹ is —O—.

12. The compound of claim 1, wherein R³ and R⁴ are independently hydrogen, optionally substituted C₁₋₆ alkyl, optionally substituted five or six membered monocyclic heterocyclyl, optionally substituted phenyl, or optionally substituted five or six membered monocyclic heteroaryl,
wherein said alkyl, heterocyclyl, aryl, or heteroaryl moiety is optionally substituted with from 1 to 3 substituents independently selected from the group consisting of halo, lower alkyl, NO₂, CF₃, CN, OR²⁰, SR²⁰, N(R²⁰)₂, SO₂R²², SO₂N(R²⁰)₂, NR²⁰COR²², COR²⁰, CO₂R²⁰, CON(R²⁰)₂, NR²⁰SO₂R²², OC(O)R²⁰, wherein R²⁰ and R²² are independently selected from the group consisting of hydrogen, C₁₋₁₅ alkyl, C₂₋₁₅ alkenyl, C₂₋₁₅ alkynyl, heterocyclyl, aryl, and heteroaryl.

13. The compound of claim 1, wherein R³ is hydrogen and R⁴ is selected from the group consisting of hydrogen, optionally substituted C₁₋₆ alkyl, optionally substituted live or six membered monocyclic heterocyclyl, optionally substituted phenyl, optionally substituted five or six membered monocyclic heteroaryl.

14. The compound of claim 1, wherein R³ is hydrogen and R⁴ is selected from the group consisting of hydrogen, methyl, ethyl, propyl, trifluoromethyl, perfluoroethyl, pyridyl, and optionally substituted phenyl.

15. The compound of claim 12, wherein R⁴ is phenyl optionally substituted with 1, 2, or 3 substituents selected from the group consisting of methyl, methoxy, ethyl, ethoxy, propyl, propoxy, trifluoromethyl, trifluoromethoxyl, perfluoroethyl, pyridyl, or C₁₋₆ alkyl.

16. The compound of claim 10, wherein X¹ is —C(O)—NR′—.

17. A pharmaceutical composition comprising a therapeutically effective amount of the compound of claim 1 or a pharmaceutically acceptable salt, ester, prodrug, or hydrate thereof.

18. A method for treating a disease or condition in a mammal that can be treated with a stearoyl-CoA desaturase inhibitory compound comprising administering to a mammal in need thereof a therapeutically effective dose of a compound of claim 1 or a pharmaceutically acceptable salt, ester, prodrug, solvate, or hydrate thereof.

19. The method of claim 18, wherein the disease state is selected from the group consisting of coronary artery disease, atherosclerosis, heart disease, hypertension, and peripheral vascular disease, cancer, cerebrovascular diseases (including, but not limited to, stroke, ischemic stroke and transient ischemic attack (TIA), and ischemic retinopathy), dyslipidemia, obesity, diabetes, insulin resistance, decreased glucose tolerance, non-insulin-dependent diabetes mellitus, Type II diabetes, Type I diabetes, and other diabetic complications.
